(19) United States
(12) Patent Application Publication
(10) Pub. No.: US 2021/0059588 A1
(43) Pub. Date: Mar. 4, 2021

(54) BODILY FLUID COLLECTION DEVICES AND RELATED METHODS

(71) Applicant: Tasso, Inc., Seattle, WA (US)

(72) Inventors: Emily Welch, Seattle, WA (US); Ellen Hayes, Seattle, WA (US); Erwin Berthier, Seattle, WA (US); Jake Myre, Seattle, WA (US); Ben Casavant, Seattle, WA (US)

(21) Appl. No.: 17/006,248

(22) Filed: Aug. 28, 2020

Related U.S. Application Data
(60) Provisional application No. 62/894,531, filed on Aug. 30, 2019.

(51) Int. Cl. A61B 5/151; A61B 5/15

(52) U.S. Cl. CPC A61B 5/15117; A61B 5/150442

(57) ABSTRACT

Devices and methods for withdrawing bodily fluid from a patient are disclosed herein. In some embodiments, a handheld device can include a housing having an opening, and a skin-piercing assembly and a plunger positioned at least partially within the housing. The skin-piercing assembly can include a blade and a biasing member, such as a torsion spring. The plunger can be movable relative to the housing from a first position to a second position. In the first position, the plunger can engage the skin-piercing assembly to maintain the biasing member in a biased configuration. Movement of the plunger from the first position to the second position can disengage the plunger from the skin-piercing assembly to permit the first biasing member to drive the blade through and/or across the opening in the base.

### CROSS-REFERENCE TO RELATED APPLICATION

This application claims the benefit of U.S. Provisional Patent Application No. 62/894,531, filed Aug. 30, 2019, and titled “BODILY FLUID COLLECTION DEVICES AND RELATED METHODS,” which is incorporated herein by reference in its entirety.

### FEDERALLY SPONSORED RESEARCH OR DEVELOPMENT

This invention was made with government support under Contract #HDTRA1-17-C-0066 awarded by the Defense Threat Reduction Agency (DTRA). The government has certain rights in the invention.

### TECHNICAL FIELD

The present technology is related to collecting bodily fluid from a patient and, in particular, to handheld bodily fluid collection devices and related methods.

### BACKGROUND

Devices, systems and methods to collect bodily fluids, such as blood, are widely used in personalized, clinical, and field medical applications. Biological samples are commonly collected using simple lancing devices or more sophisticated devices that require trained personnel (e.g., phlebotomy venipunctures). Transferring bodily fluids to a container, receptacle, or an analysis device often requires several steps, which can be time consuming, prone to error, and/or cumbersome. Moreover, many personalized devices designed for untrained users can obtain only very limited volumes of bodily fluid, which in turn limits the applicability of such devices.

### BRIEF DESCRIPTION OF THE DRAWINGS

Many aspects of the present technology can be better understood with reference to the following drawings. The components in the drawings are not necessarily to scale. Instead, emphasis is placed on clearly illustrating the principles of the present technology.

FIG. 1A is a perspective view of a bodily fluid collection device configured in accordance with embodiments of the present technology; FIG. 1B is a perspective view of the bodily fluid collection device during use; and FIG. 1C is a perspective view illustrating detachment of a collection reservoir from the bodily fluid collection device.

FIG. 2 is a partially-schematic side cross-sectional view of the bodily fluid collection device of FIGS. 1A-1C in accordance with embodiments of the present technology.

FIGS. 3A-3D are partially-schematic side cross-sectional views of the bodily fluid collection device of FIGS. 1A-1C illustrating various stages of a procedure to withdraw bodily fluid from a patient in accordance with embodiments of the present technology.

FIG. 4 is a top cross-sectional view of the bodily fluid collection device of FIGS. 1A-1C including a fluidic channel for directing bodily fluid to the collection reservoir in accordance with embodiments of the present technology.

FIGS. 5A and 5B are top cross-sectional views of the bodily fluid collection device of FIGS. 1A-1C including microfluidic channels for directing bodily fluid to the collection reservoir in accordance with embodiments of the present technology.

FIGS. 6A-6C are partially-schematic side cross-sectional views of the bodily fluid collection device of FIGS. 1A-1C including a flexible membrane and illustrating various stages of a procedure to withdraw bodily fluid from a patient in accordance with embodiments of the present technology.

FIG. 7 is a partially-schematic side cross-sectional view of a bodily fluid collection device configured in accordance with additional embodiments of the present technology.

FIG. 8A is a side cross-sectional view of a bodily fluid collection device configured in accordance with additional embodiments of the present technology; FIG. 8B is a side view of a skin-piercing assembly of the bodily fluid collection device; and FIG. 8C is a rear view of a retaining feature and a base portion of the bodily fluid collection device.

FIGS. 9A-9E are partially-schematic side cross-sectional views of the bodily fluid collection device of FIGS. 8A-8C illustrating various stages of a procedure to withdraw bodily fluid from a patient in accordance with embodiments of the present technology.

FIG. 10 is a side cross-sectional view of a bodily fluid collection device configured in accordance with additional embodiments of the present technology.

FIGS. 11A and 11B are partial cross-sectional side views of a bodily fluid collection device in a pre-deployment configuration and a deployed configuration, respectively, configured in accordance with additional embodiments of the present technology; FIG. 11C is a top view of a housing of the bodily fluid collection device of FIGS. 11A and 11B; and FIG. 11D is a side cross-sectional view of the housing taken along the line 11D in FIG. 11C.

FIGS. 12A-12C are side cross-sectional views of a lumen of the bodily fluid collection device of FIGS. 11A and 11B taken along the line 12A in FIG. 11A and illustrating the bodily fluid collection device in the pre-deployment position, a mid-deployment position, and the deployed position, respectively, in accordance with embodiments of the present technology.

FIG. 13A is a partially transparent side view of a skin-piercing assembly coupled to a portion of a housing of a bodily fluid collection device configured in accordance with additional embodiments of the present technology.

FIGS. 14A-14C are side views of the skin-piercing assembly of FIG. 13A in a pre-deployment position, a mid-deployment position, and a deployed position, respectively, in accordance with embodiments of the present technology.

FIGS. 15A and 15B are a perspective side view and a cross-sectional side view, respectively, of a skin-piercing assembly coupled to a portion of a housing of a bodily fluid collection device and configured in accordance with additional embodiments of the present technology.

FIG. 15C is a side cross-sectional view of the housing of FIGS. 15A and 15B in accordance with embodiments of the present technology.

### DETAILED DESCRIPTION

The present technology is directed generally to devices and methods for deploying a skin-piercing feature toward/into a patient's skin to withdraw and collect bodily fluid (e.g., blood). In some embodiments, a device for withdrawing bodily fluid from a patient can include a housing including a base configured to be positioned against the skin of the patient. The base can include an opening extending therethrough for collecting the bodily fluid. A skin-piercing assembly and a plunger can be positioned at least partially within the housing. The skin-piercing assembly can include a drive member, a skin-piercing feature (e.g., a blade) coupled to the drive member, and a biasing member coupled to the drive member. The plunger can be configured to move relative to the housing from a first position to a second position. In the first position, the plunger can engage the drive member of the skin-piercing assembly to maintain the first biasing member in a biased configuration. When the first biasing member is in the biased configuration, the skin-piercing feature can be rotated away from the opening in the base. However, movement of the plunger from the first position to the second position can disengage the plunger from the drive member to permit the first biasing member to drive the skin-piercing feature at least partially through the opening in the base to incise the skin of the subject. In some aspects of the present technology, the device can be used to quickly and easily obtain a volume of bodily fluid sufficient for downstream testing and analysis.

In some embodiments, the base of the housing is configured to seal against the skin of the subject. The device can further include a sealing member operably coupled to the plunger such that movement of the plunger from the first position to the second position increases a sealed volume within the housing to generate vacuum pressure. In some embodiments, the vacuum pressure can be generated before the plunger disengages the skin-piercing assembly and the skin-piercing feature is driven into the skin. In such embodiments, the vacuum pressure can draw the skin at least partially into the opening to, for example, increase the volume of bodily fluid withdrawn. In other embodiments, the vacuum pressure can be generated during and/or after the plunger disengages the skin-piercing assembly and the skin-piercing feature is driven into the skin.

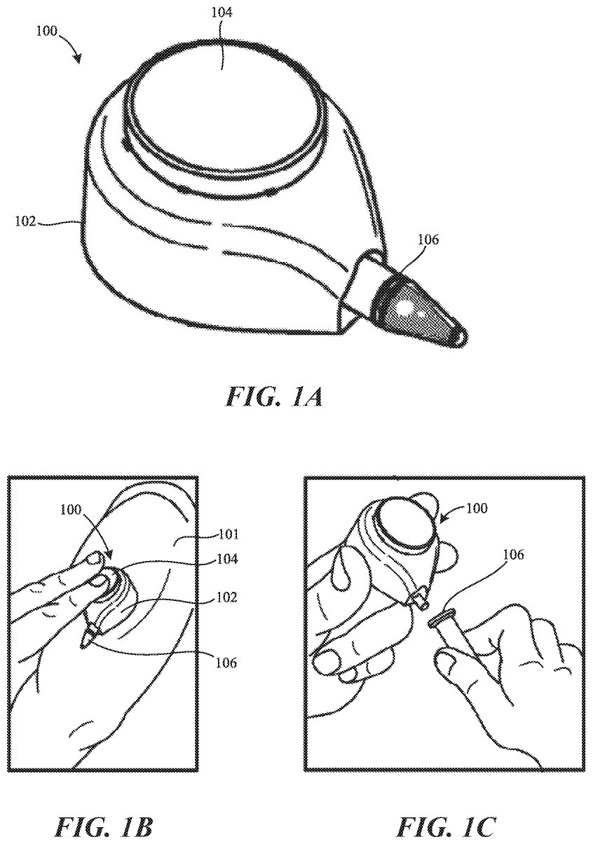

Specific details of several embodiments of the present technology are described herein with reference to FIGS. 1A-15C. However, the present technology may be practiced without some of these specific details. In some instances, well-known structures and techniques often associated with bodily fluid collection devices have not been shown in detail so as not to obscure the present technology. The terminology used in the description presented below is intended to be interpreted in its broadest reasonable manner, even though it is being used in conjunction with a detailed description of certain specific embodiments of the disclosure. Certain terms may even be emphasized below; however, any terminology intended to be interpreted in any restricted manner will be overtly and specifically defined as such in this Detailed Description section.

The accompanying Figures depict embodiments of the present technology and are not intended to be limiting of its scope. The sizes of various depicted elements are not necessarily drawn to scale, and these various elements may be arbitrarily enlarged to improve legibility. Component details may be abstracted in the Figures to exclude details such as position of components and certain precise connections between such components when such details are unnecessary for a complete understanding of how to make and use the present technology. Many of the details, dimensions, angles, and other features shown in the Figures are merely illustrative of particular embodiments of the disclosure. Accordingly, other embodiments can have other details, dimensions, angles, and features without departing from the spirit or scope of the present technology.

FIG. 1A is a perspective view of a bodily fluid collection device 100 (“device 100”) configured in accordance with embodiments of the present technology. The device 100 can be handheld with a size that is easily grasped and manipulated by one or both of a patient's hands. Such handheld devices advantageously allow a patient to collect a bodily fluid sample (e.g., a blood sample) without assistance from another individual. In some embodiments, the handheld devices of the present technology can be operated by a layperson outside of a medical setting (e.g., at home or in a field clinic) and without the aid of a medical professional.

In the illustrated embodiment, the device 100 includes a housing 102 and an actuator 104. The actuator 104 (e.g., a button) can be movable relative to the housing 102 to actuate/initiate withdrawal of a bodily fluid from the patient. The housing 102 is removably coupled to a collection reservoir 106 (e.g., a tube or cartridge) for receiving the bodily fluid withdrawn from the patient. The reservoir 106 can act as a removable and standardized container for bodily fluid that can be detached and inserted into clinical and laboratory equipment or workflows (e.g., for diagnostics and/or biomarker detection).

FIG. 1B is a perspective view of the bodily fluid collection device 100 in use by the patient. To collect a bodily fluid sample, the device 100 is applied to a patient's body, with a bottom surface of the housing 102 positioned against the skin 101 of the patient and the actuator 104 positioned away from the skin 101. Actuating (e.g., pressing, twisting, pulling) the actuator 104 deploys a skin-piercing feature (e.g., a blade, lancet) from within the device 100 to pierce the skin 101. In some embodiments, the device 100 is configured to generate a vacuum within the device 100 that acts against the patient's skin either directly or indirectly, and before and/or after deployment of the skin-piercing feature. Bodily fluid from the resulting incision is withdrawn into the housing 102 and collected into the reservoir 106.

FIG. 1C is a perspective view illustrating detachment of the reservoir 106 from the device 100. Once the desired amount of bodily fluid has been collected into the reservoir 106, the device 100 is removed from the skin 101, and the reservoir 106 is detached from the housing 102.

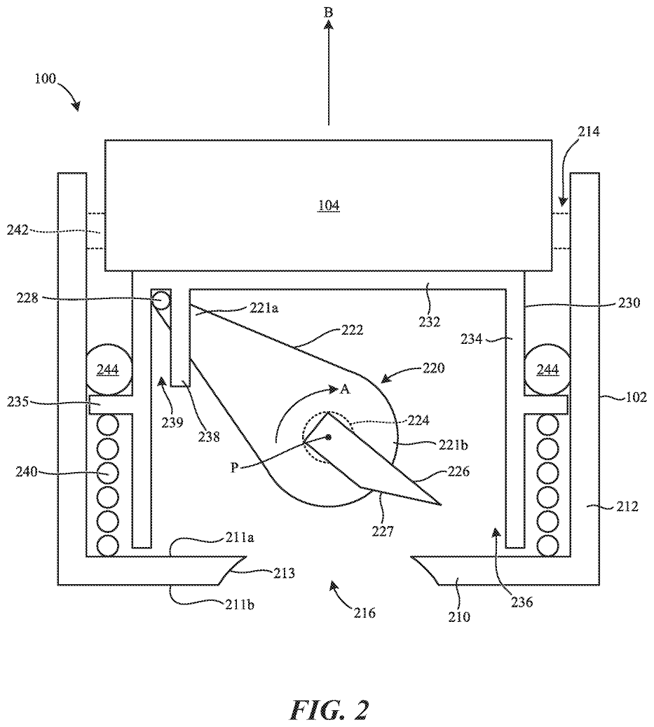

FIG. 2 is a partially schematic side cross-sectional view of the device 100 of FIGS. 1A-1C in accordance with embodiments of the present technology. The device 100 is in a pre-deployed configuration in FIG. 2. The device 100 includes the housing 102, the actuator 104, a skin-piercing assembly 220, and a plunger 230 (e.g., a platform, release member, inner housing). In the illustrated embodiment, the housing 102 includes a base portion 210 and a sidewall portion 212 extending from the base portion 210 (e.g., extending generally perpendicular to and upward away from the base portion 210). The base portion 210 and the sidewall portion 212 together define a lumen 214 (e.g., an opening, cavity) in which the skin-piercing assembly 220 and the plunger 230 are at least partially positioned. The base portion 210 further includes an upper surface 211a facing (e.g., open to) the lumen 214 and a lower surface 211b opposite the upper surface 211a. During use of the device 100, the lower surface 211b is configured to be positioned against and/or adjacent to the skin 101 (FIG. 1B) of the patient.

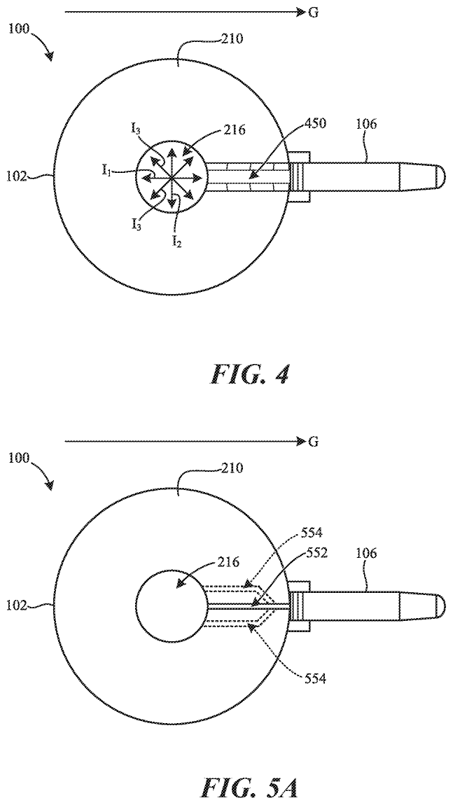

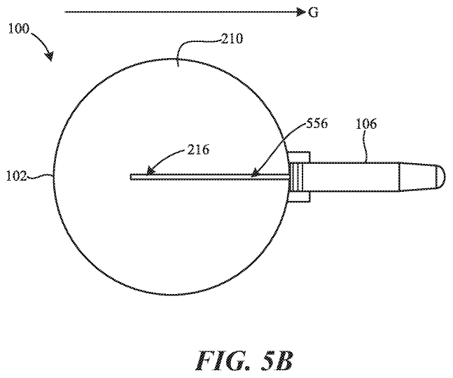

An opening 216 (e.g., an aperture, a collection site) can extend through the base portion 210 between the upper and lower surfaces 211a and 211b such that the opening 216 abuts the skin 101 of the patient during use of the device 100. In some embodiments, the opening 216 can have a cross-sectional dimension (e.g., width, area) that varies between the upper and lower surfaces 211a and 211b to, for example, facilitate drawing of the skin 101 of the patient into and/or toward the lumen 214 during use of the device 100. For example, in the illustrated embodiment a portion 213 (e.g., a sidewall) of the base portion 210 that abuts/defines the opening 216 has a shape/profile that curves inward in a direction from the lower surface 211b toward the upper surface 211a. In other embodiments, the portion 213 of the base portion 210 can have other shapes/profiles, such as a linear profile that slopes inward, a curved profile that curves outward, a linear profile that slopes outward, or a generally vertical profile. In some embodiments, as described in detail with reference to FIGS. 4 and 5, the opening 216 can be fluidly connected to a fluidic channel (e.g., a microfluidic channel) formed in, on, and/or through the base portion 210. During use of the device 100, the fluidic channel can direct bodily fluid from the opening 216 to the reservoir 106 (FIGS. 1A-1C).

In the illustrated embodiment, the skin-piercing assembly 220 includes a drive member 222 coupled to a first biasing member 224 (obscured in FIG. 2 and therefore shown in broken lines). In some embodiments, the first biasing member 224 couples the drive member 222 to the housing 102 such that the drive member 222 is rotatably/pivotably mounted within the lumen 214 of the housing 102. For example, the first biasing member 224 can be a torsion spring or other suitable biasing member that is connected between the drive member 222 and the sidewall portion 212 of the housing 102 and/or another portion of the housing 102. More specifically, the drive member 222 can include a generally elongate body extending between a first portion 221a and a second portion 221b, and the first biasing member 224 can be coupled to or near to the second portion 221b of the drive member 222 such that the drive member 222 is pivotable about a pivot axis P (e.g., in a direction indicated by arrow A).

The skin-piercing assembly 220 can further include (i) a skin-piercing feature 226 coupled to the drive member 222 at or near the second portion 221b of the drive member 222 and (ii) a release member 228 (e.g., a tab, bar, projection) coupled at or near the first portion 221a of the drive member 222. In the illustrated embodiment, the skin-piercing feature 226 is a blade having a sharpened cutting edge 227. In other embodiments, the skin-piercing feature 226 is a needle, lancet (e.g., cylindrical or other shaped lancet), or other feature configured to pierce the skin 101 (FIG. 1B) of the patient. In some embodiments, the skin-piercing assembly 220 can include multiple skin-piercing features (e.g., multiple offset blades to produce a varied cutting pattern). The release member 228 is configured to engage the plunger 230 to maintain the skin-piercing assembly 220 in a biased (e.g., wound) configuration shown in FIG. 2. More specifically, in the illustrated embodiment the plunger 230 includes a base portion 232 (e.g., an upper portion) and a sidewall portion 234 extending from the base portion 232 (e.g., extending generally perpendicular to and downward away from the base portion 232). In some embodiments, the base portion 232 and the sidewall portion 234 can at least partially define a lumen 236 in which the skin-piercing assembly 220 is positioned. The plunger 230 can further include a restraint portion 238 (e.g., a projection, flange) extending downward away from the base portion 232 and defining a channel 239 together with the sidewall portion 234. In the illustrated embodiment, when the skin-piercing assembly 220 is in the biased configuration, the release member 228 of the skin-piercing assembly 220 is positioned at least partially within the channel 239 such that the restraint portion 238 of the plunger 230 engages the release member 228 and inhibits the drive member 222 (and the skin-piercing feature 226) from pivoting about the pivot axis P in the direction indicated by the arrow A.

In the illustrated embodiment. the plunger 230 is operably coupled to (i) the actuator 104 and (ii) a second biasing member 240 (e.g., a compression spring) configured to drive the plunger 230 through the lumen 214 away from the base portion 210 of the housing 102 (e.g., in the direction indicated by arrow B). For example, the base portion 232 of the plunger 230 can be coupled to the actuator 104. Likewise, the plunger 230 can include a flange portion 235 projecting outwardly away from the sidewall portion 234, and the second biasing member 240 can be coupled between the flange portion 235 and the base portion 210 of the housing 102. In other embodiments, the plunger 230 can be operably coupled to the second biasing member 240 in other manners. For example, the flange portion 235 can project inward from the sidewall portion 234 of the plunger 230 such that the second biasing member 240 is positioned at least partially within the lumen 236 of the plunger 230, or the second biasing member 240 can extend between the base portion 232 of the plunger 230 and the housing 102.

In the illustrated embodiment, the second biasing member 240 is in a compressed/biased configuration and therefore exerts a biasing force against the plunger 230. Accordingly, when the device 100 is in the pre-deployed shown in FIG. 2, both of the first and second biasing members 224, 240 are in a biased state. To maintain/lock the device 100 in the pre-deployed configuration, the device 100 can further include a lock mechanism 242 (shown schematically) configured to lock the position of the plunger 230 within the housing 102. The actuator 104 can be operably coupled to the lock mechanism 242 and configured such that actuation of actuator 104 selectively unlocks the lock mechanism 242 to permit the second biasing member 240 to drive the plunger 230 upward in the direction indicated by the arrow B. Accordingly, in some embodiments the actuator 104 can be referred to as a release member, a release actuator, or a release mechanism. In some embodiments, pressing and/or pulling the actuator 104 upward and/or downward can unlock the lock mechanism 242 and release the plunger 230. In some embodiments, the lock mechanism 242 can include one or more engagement features (e.g., flanges, grooves) positioned on/between the actuator 104 and the housing 102 (e.g., an inner surface of the sidewall portion 212 of the housing). Accordingly, for example, twisting the actuator 104 and/or translating the actuator 104 within the lumen 214 could unlock the actuator 104 and the plunger 230 operably coupled thereto. In other embodiments, the actuator 104 can be slidably coupled to the housing 102 such that the actuator 104 can be slid (e.g., horizontally) out of engagement with the plunger 230 to thereby release the plunger 230. In yet other embodiments, the lock mechanism 242 can be a mechanical or electrical switch.

In the illustrated embodiment, the device 100 further includes a sealing member 244 positioned between the sidewall portion 234 of the plunger 230 and the sidewall portion 212 of the housing 102. The sealing member 244 can comprise an O-ring, lip seal, quad ring, rolling membrane, or the like, and is configured to seal the interface between the plunger 230 and the housing 102. In some embodiments, the flange portion 235 of the plunger 230 engages the sealing member 244 such that the sealing member 244 is driven upward (e.g., in the direction of the arrow B) by the plunger 230 when the lock mechanism 242 is unlocked to, for example, generate a vacuum within a portion of the lumen 214 of the housing 102 adjacent the opening 216.

The various components of the device 100 can comprise metal, plastic, and/or other suitable materials. For example, in some embodiments the housing 102, the actuator 104, the plunger 230, the drive member 222, and/or other components of the device 100 be 3D-printed, molded (e.g., injection molded), or otherwise formed from a plastic material. In some embodiments, the device 100 can be manufactured to have the pre-deployed configuration shown in FIG. 2.

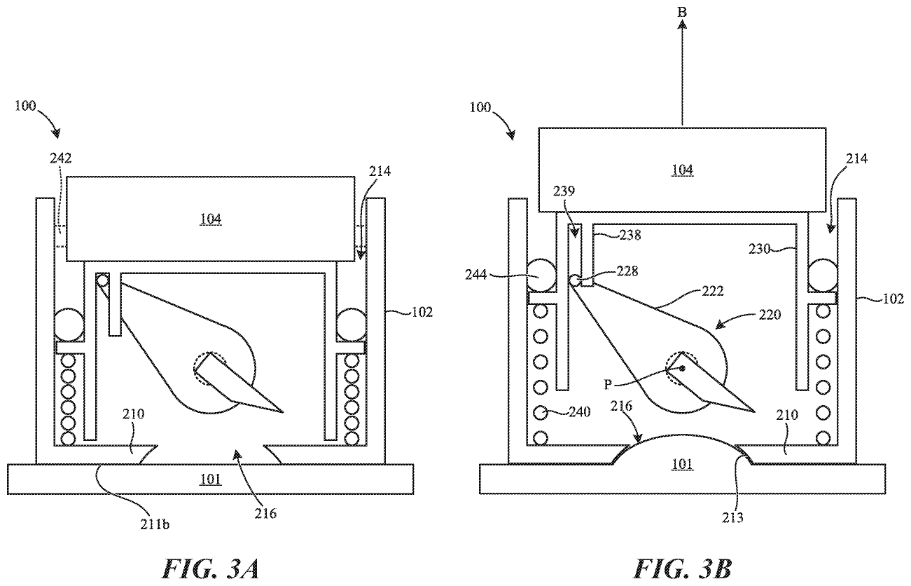

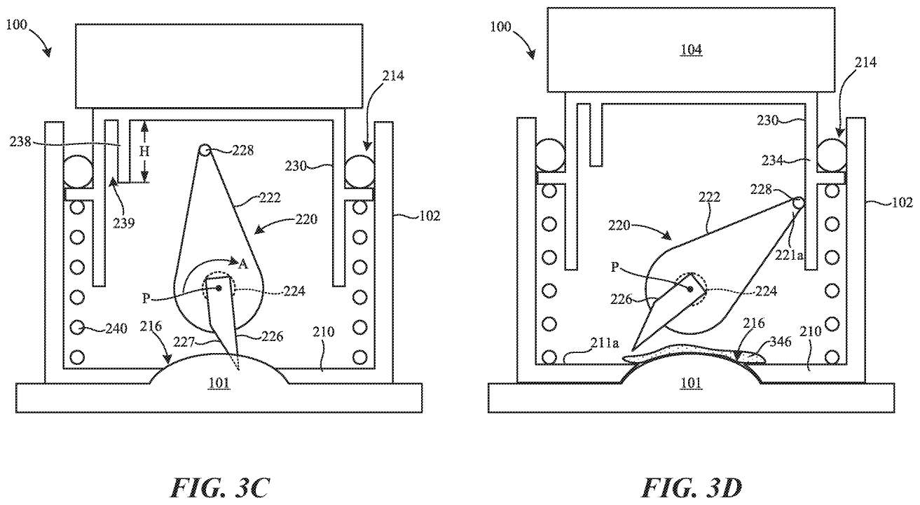

FIGS. 3A-3D are side cross-sectional views of the device 100 illustrating various stages of a procedure to withdraw bodily fluid (e.g., blood) from a patient in accordance with embodiments of the present technology. Referring first to FIG. 3A, the device 100 is initially placed against the skin 101 of the patient in the pre-deployed configuration (e.g., a first configuration, an initial configuration, a biased configuration) shown in FIG. 2. More particularly, the lower surface 211b of the base portion 210 of the housing 102 can be positioned against the skin 101. In some embodiments, the base portion 210 of the housing 102 can form a seal with the skin 101 of the patient such that (i) the opening 216 of the housing 102 is sealed off from the environment around the device 100 and (ii) the device 100 includes a sealed volume within a portion of the lumen 214 of the housing 102.

FIG. 3B shows the device 100 in a partially-deployed configuration (e.g., a second configuration) after the actuator 104 has been actuated to unlock the lock mechanism 242 (FIG. 3A) and release the plunger 230. In the illustrated embodiment, the second biasing member 240 drives the plunger 230 upward (e.g., in the direction of the arrow B) through the lumen 214 and away from the base portion 210 of the housing 102. The plunger 230 is also driven upward relative to the skin-piercing assembly 220 such that the restraint portion 238 of the plunger 230 moves relative to the release member 228 of the skin-piercing assembly 220. That is, as the plunger 230 moves upward relative to the skin-piercing assembly 220, the release member 228 moves from a position near an upper end portion of the channel 239 to a position near a lower end portion of the channel 239. Nevertheless, in the partially-deployed configuration shown in FIG. 3B, the restraint portion 238 of the plunger 230 still engages the release member 228 of the skin-piercing assembly 220 such that the skin-piercing assembly 220 maintains the biased configuration and the drive member 222 does not pivot about the pivot axis P.

In the illustrated embodiment, the second biasing member 240 also drives the sealing member 244 upward through the housing 102—thereby increasing the sealed volume within the lumen 214 of the housing 102. In some aspects of the present technology, increasing the sealed volume within the lumen 214 generates reduced pressure (e.g., vacuum pressure) therein. In some embodiments, the reduced pressure can draw the skin 101 at least partially into the opening 216 as shown in FIG. 3B. That is, the reduced pressure can deflect the skin 101 into the opening 216 and/or into the lumen 214 of the housing 102. In some embodiments, the portion 213 of the base portion 210 that abuts/defines the opening 216 has a shape/profile that facilitates the movement of the skin 101 into the opening 216.

FIG. 3C shows the device 100 in a deployed configuration (e.g., a third configuration, an incision configuration) after continued upward movement of the plunger 230 within the lumen 214 of the housing 102. In the illustrated embodiment, the second biasing member 240 has driven the plunger 230 upward (e.g., in the direction of the arrow B) through the lumen 214 until the restraint portion 238 of the plunger 230 is spaced apart from and no longer engages the release member 228 of the skin-piercing assembly 220. That is, a relaxed length of the second biasing member 240 can be long enough to enable the second biasing member 240 to drive the plunger 230 upward until the release member 228 is no longer positioned within the channel 239. When the skin-piercing assembly 220 is no longer restrained by the plunger 230, the first biasing member 224 drives the drive member 222 to pivot about the pivot axis P (e.g., in the direction of the arrow A). This movement of the drive member 222 pivots the skin-piercing feature 226 downward toward/into the opening 216 and the skin 101. As shown, all or a portion of the cutting edge 227 can move through the opening 216 and can contact and incise the skin 101 positioned therein. In some aspects of the present technology, the release member 228 is released from within the channel 239 instantaneously or nearly instantaneously, which can increase the velocity of the skin-piercing feature 226 to, facilitate for example, a cleaner incision, reduced patient pain, and/or a larger draw volume.

The position of the skin-piercing assembly 220 relative to the opening 216 and the relative size and/or shape of the skin-piercing feature 226 can be varied to vary the size of the incision. For example, the device 100 can be configured to generate an incision in the skin 101 that is: about 3 millimeters long and about 2 millimeters deep, or about 5 millimeters long and about 1 millimeter deep. Moreover, a height H of the restraint portion 238 of the plunger 230 can be selected to provide a desired level of vacuum before release and triggering of the skin-piercing assembly 220. For example, increasing the height H can increase the size of the sealed volume in the lumen 214 of the housing 102—and thus the vacuum pressure generated—before the skin-piercing assembly 220 is moved out of engagement with the plunger 230 and triggered to rotate. Conversely, decreasing the height H can cause more of the vacuum pressure to be generated during and/or after the skin-piercing feature 226 incises the skin 101. For example, in some embodiments the height H can be selected to be just large enough to secure the skin-piercing assembly 220 in the pre-deployed configuration (FIG. 3A). In such embodiments, the plunger 230 rapidly disengages the skin-piercing assembly 220 as it is driven upward such that substantially all the vacuum pressure is generated after the skin-piercing assembly 220 begins to pivot.

FIG. 3D shows the device 100 in a retracted configuration (e.g., a fourth configuration) after continued rotation of the skin-piercing assembly 220 within the lumen 214 of the housing 102. In the illustrated embodiment, the first biasing member 224 has driven the drive member 222 around the pivot axis P until (i) the skin-piercing feature 226 has swept past the opening 216 and no longer contacts the skin 101 of the patient and (ii) the first portion 221a of the drive member 222 (e.g., the release member 228) contacts the sidewall portion 234 of the plunger 230. In other embodiments, the drive member 222 can be configured (e.g., shaped and sized) to rotate further about the pivot axis P such that, for example, the drive member 222 does not contact the plunger 230 in the retracted configuration. Moreover, in the retracted configuration, the skin-piercing assembly 220 is spaced vertically apart from the base 210 of the housing 102. In some aspects of the present technology, such a gap can facilitate movement of the bodily fluid 346 into the device 100.

As shown in FIG. 3D, incising the skin 101 causes bodily fluid 346 (e.g., blood) to flow from the skin 101 into the device 100—for example, into the lumen 214 of the housing 102 and/or onto the upper surface 211a of the base portion 210 of the housing 102. In some aspects of the present technology, the vacuum pressure generated within the device 100 prior to cutting the skin 101 with the skin-piercing feature 226 can increase capillary action within the skin 101 and thus increase the amount of the bodily fluid 346 withdrawn/collected. Similarly, the high rotational velocity of the skin-piercing feature 226 can increase the amount of the bodily fluid 346 withdrawn/collected while also making the procedure relatively painless for the patient.

The amount of the bodily fluid 346 withdrawn into the device 100, also known as the “draw volume,” can be sufficiently large for downstream testing and analysis of the bodily fluid 346 for, for example, diagnostics and/or biomarker detection performed on a blood sample. As used herein, draw volume can refer to the maximum volume of bodily fluid that can be collected from a specified percentage of the patient population, for example, from at least 90% of patients. In some embodiments, the draw volume of the device 100 can be between 100-1000 μL (e.g., between about 600-700 μL). In another aspect of the present technology, the device 100 is configured to draw the bodily fluid 346 in a relatively short period of time compared to conventional devices. For example, in some embodiments the device 100 can collect the draw volume in less than about 1 minute, less than about 45 seconds, less than about 30 seconds, or less than about 15 seconds.

In some embodiments, the device 100 is configured as a single-use device. For example, in the illustrated embodiment the device 100 is configured such that actuation of the actuator 104 when the device 100 is in the retracted configuration does not pivot the skin-piercing feature 226 into the opening 216. Specifically, the first biasing member 224 is no longer biased in the retracted configuration and therefore cannot drive the skin-piercing feature 226 toward the opening 216. In other embodiments, the device 100 can have other features specifically configured to limit the device 100 to a single use. For example, the actuator 104 can be configured as a pass-through actuator that does not reengage the skin-piercing assembly 220 after use.

In some embodiments, the device 100 can include one or more fluidic features configured to facilitate the transfer/movement of the bodily fluid 346 from the opening 216 to the reservoir 106 (FIGS. 1A-1C). For example, FIGS. 4-5B are top cross-sectional views of the housing 102 and the reservoir 106 and illustrating various fluidic features for directing the bodily fluid 346 from the opening 216 to the reservoir 106 in accordance with embodiments of the present technology.

Referring to FIG. 4, the device 100 can include a fluidic channel 450 formed in and/or on the base portion 210 of the housing 102 and configured to fluidly couple the opening 216 of the housing 102 to the reservoir 106. In some embodiments, during use of the device 100, the device 100 can be aligned with the gravitational field (e.g., as indicated by arrow G) such that the reservoir 106 is positioned below the opening 216. Accordingly, in some embodiments the fluidic channel 450 is configured (e.g., sized and shaped) to direct the bodily fluid 346 (FIG. 3D) from the opening 216 to the reservoir 106 via the force of gravity. In some such embodiments, the fluidic channel 450 is not configured to impart any capillary force on the bodily fluid 346. In some aspects of the present technology, the fluidic channel 450 imparts substantially no shear force on the bodily fluid 346—which can improve the quality of the bodily fluid 346 for testing purposes (e.g., diagnostic blood tests). In some embodiments, all or a portion of the fluidic channel 450 can be sloped—for example, sloping upward away from the opening 216 toward the reservoir 106. In some embodiments, the slope can be selected such that the gravitational force is still great enough to drive the bodily fluid 346 from the opening 216 to the reservoir 106. In some embodiments, the base portion 210 of the housing 102 can be coated with a hydrophobic (e.g., superhydrophobic) material that helps promote the flow of the bodily fluid 346 from the opening 216 toward the reservoir 106 without capillary forces.

Referring to FIGS. 2-4 together, the skin-piercing assembly 220 can be configured (e.g., sized, shaped, and positioned) to sweep across/through the opening 216 in any direction to create an incision in the skin 101 of the patient that has any selected orientation relative to the fluidic channel 450. For example, the skin-piercing assembly 220 can be configured to incise the skin 101 of the patient in a direction generally parallel to the fluidic channel 450 (e.g., as indicated by arrow I₁ in FIG. 4), in a direction generally perpendicular to the fluidic channel 450 (e.g., as indicated by arrow 12 in FIG. 4), and/or in a direction that is angled relative to the fluidic channel 450 (e.g., as indicated by arrows 13 in FIG. 4). In the illustrated embodiment, the opening 216 has a generally circular cross-sectional shape. In other embodiments, the opening 216 can have other cross-sectional shapes (e.g., rectilinear, polygonal, irregular) and/or can have a different size.

Referring to FIG. 5A, the device 100 can include a microfluidic channel 552 formed in and/or on the base portion 210 of the housing 102 and configured to fluidly couple the opening 216 of the housing 102 to the reservoir 106. In some embodiments, the microfluidic channel 552 is an open channel (e.g., including a base surface, sidewalls, and an elongate opening over/opposite the base surface) configured to impart a capillary force on the bodily fluid 346 (FIG. 3D). The microfluidic channel 552 can be of the type described in detail in (i) U.S. patent application Ser. No. 13/949,108, filed Jul. 23, 2013, and titled “METHODS, SYSTEMS, AND DEVICES RELATING TO OPEN MICROFLUIDIC CHANNELS”, and/or (ii) U.S. patent application Ser. No. 14/816,994, filed Aug. 3, 2015, and titled “DEVICES, SYSTEMS AND METHODS FOR GRAVITY-ENHANCED MICROFLUIDIC COLLECTION, HANDLING AND TRANSFERRING OF FLUIDS”, both of which are incorporated herein by reference in their entireties. In some embodiments, during use of the device 100, the device 100 can be aligned with the gravitational field (e.g., as indicated by arrow G) such that the reservoir 106 is positioned below the opening the opening 216. Accordingly, in some embodiments the microfluidic channel 552 is configured (e.g., sized and shaped) to direct the bodily fluid 346 from the opening 216 to the reservoir 106 via a capillary force, the force of gravity, or both a capillary force and the force of gravity.

In some embodiments, the device 100 can include one or more additional microfluidic channels 554 (shown in phantom) configured to direct the bodily fluid 346 from the opening 216 to the reservoir 106. That is, for example, the device 100 can include a microfluidic network configured to direct the bodily fluid 346 to the reservoir 106. Likewise, referring to FIGS. 2-3D and 5A together, the skin-piercing assembly 220 can be configured (e.g., sized, shaped, and positioned) to sweep across/through the opening 216 in any direction to create an incision in the skin 101 of the patient that has any desired orientation relative to the microfluidic channel 552 and/or the microfluidic channels 554.

Referring to FIG. 5B, the opening 216 can be a portion of a microfluidic channel 556 extending through/along the base portion 210 of the housing 102 to the reservoir 106. Referring to FIGS. 2-3D and 5B together, the skin-piercing assembly 220 can be configured (e.g., sized, shaped, and positioned) to sweep across/through the opening 216 along a length of the microfluidic channel 556 to create an incision in the skin 101. Accordingly, the microfluidic channel 556 can extend entirely through the base portion 210 and can be defined by opposing sidewalls at least where the blade 226 is configured to sweep through the microfluidic channel 556. In some embodiments, the microfluidic channel 556 is configured to impart a capillary force on the bodily fluid 346 (FIG. 3D). In some aspects of the present technology, incising the skin 101 within a microfluidic channel 556 can decrease the time required for the bodily fluid 346 to flow from the incision to the reservoir 106.

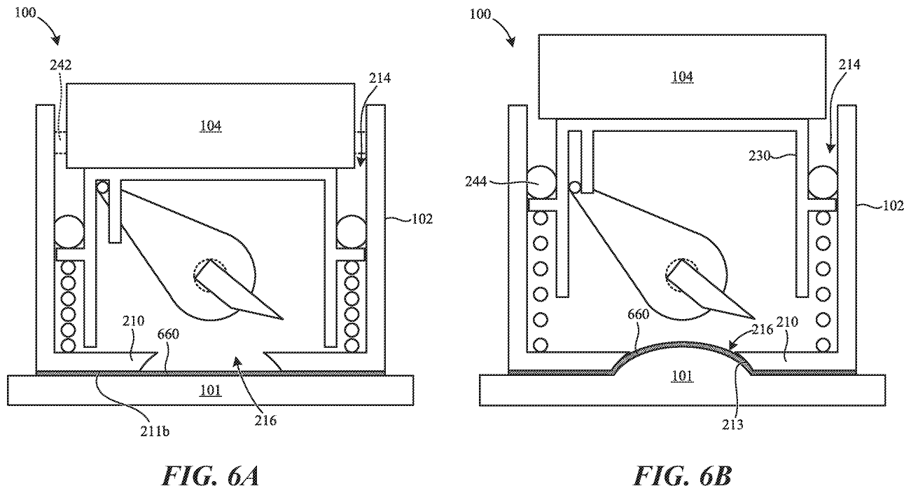

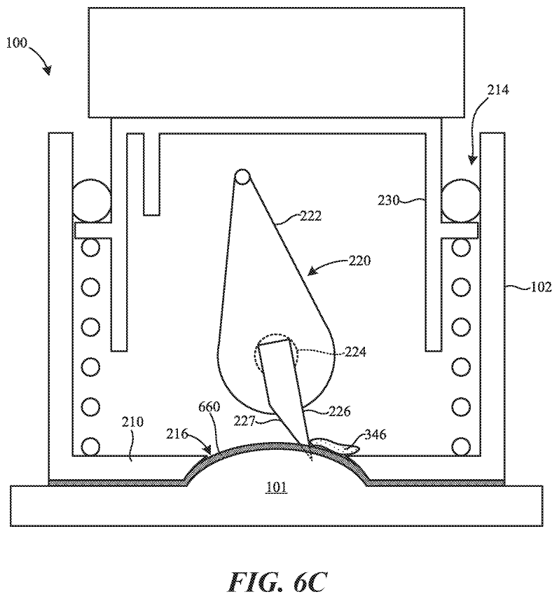

FIGS. 6A-6C are side cross-sectional views of the device 100 including a flexible membrane 660 and illustrating various stages of a procedure to withdraw bodily fluid (e.g., blood) from a patient in accordance with additional embodiments of the present technology. Referring first to FIG. 6A, the flexible membrane 660 can be attached to the lower surface 211b of the base portion 210 of the housing 102 and can span laterally across the opening 216. The flexible membrane 660 can be bendable and/or stretchable (e.g., elastic). For example, the flexible membrane 660 can comprise polyurethane, silicone, and/or other suitably elastic materials. The flexible membrane 660 can seal the lumen 214 of the housing 102 to create a sealed volume within the housing 102. Accordingly, the device 100 can be fully-sealed prior to use. In some embodiments, the flexible membrane 660 can be relatively thin—for example, having a thickness of less than or equal to about 250 or between about 50-400 In some embodiments, the flexible membrane 660 can be of the type described in detail in U.S. patent application Ser. No. 16/571,028, filed Sep. 13, 2019, and titled “BODILY FLUID COLLECTION DEVICES AND RELATED METHODS”, which is incorporated herein by reference in its entirety.

As shown in FIG. 6A, the device 100 is initially placed against the skin 101 of the patient in the pre-deployed configuration with the device 100 fully-sealed. More particularly, a lower surface of the flexible membrane 660 can be positioned against the skin 101. In some embodiments, the flexible membrane 660 contacts and couples to the skin 101 to provide an airtight seal against the skin 101. An adhesive (not shown) may be applied to the bottom surface of the flexible membrane 660 to facilitate sealing against the skin 101.

FIG. 6B shows the device 100 in the partially-deployed configuration after the actuator 104 has been actuated to unlock the lock mechanism 242 (FIG. 6A) and release the plunger 230. In the illustrated embodiment, the second biasing member 240 also drives the sealing member 244 upward through the housing 102 to generate reduced pressure (e.g., vacuum pressure) within the lumen 214 of the housing 102. The vacuum pressure within the housing 102 can pull the flexible membrane 660 at least partially into the opening 216 and/or the lumen 214 of the housing 102 such that that the flexible membrane 660 assumes a curved shape. Due to the seal between the skin 101 and the flexible membrane 660, the skin 101 is also pulled toward, into, and/or through the opening 216 and assumes a curvature similar to the curvature of the flexible membrane 660. The flexible membrane 660 can therefore control the curvature of the skin 101. In some embodiments, the portion 213 of the base portion 210 that abuts/defines the opening 216 has a shape that facilitates the movement of the flexible membrane 660 and the skin 101 into the opening 216.

FIG. 6C shows the device 100 in the deployed configuration after the first biasing member 224 has driven the drive member 222 to pivot the skin-piercing feature 226 toward the opening 216. This movement of the drive member 222 pivots the skin-piercing feature 226 downward toward/into the flexible membrane 660 and the skin 101 in the opening 216. As shown, all or a portion of the cutting edge 227 can sweep through and incise the flexible membrane 660 and the skin 101 positioned in the opening 216. In other embodiments, the flexible membrane 660 can optionally include an aperture to allow the skin-piercing feature 226 to pass therethrough. In some aspects of the present technology, the flexible membrane 660 is expected to provide improved control over larger areas of skin, thus allowing the device 100 to access more capillaries and increase the volume of the bodily fluid 346 that can be withdrawn. The flexible membrane 660 can also provide a support to collect the bodily fluid 346 close to the incision point to prevent or at least mitigate the bodily fluid 346 from travelling on/along the skin 101 of the patient. In some aspects of the present technology, the flexible membrane 660 can cause the bodily fluid 346 to travel more rapidly from the wound of the patient to the reservoir 106 (FIGS. 1A-1C) as compared to, for example, the device without the flexible membrane 660. In some embodiments, the flexible membrane 660 can permit the delivery of an anticoagulant material rapidly after the bodily fluid 346 is extracted from the capillaries. In some embodiments, the base portion 210 of the housing 102 and/or the flexible membrane 660 (e.g., an upper surface of the flexible membrane 660 positioned within the opening 216) can be coated with a hydrophobic (e.g., superhydrophobic) material that helps promote the flow of the bodily fluid 346 from the opening 216 toward the reservoir 106 (FIGS. 1A-1C) without capillary forces.

In other embodiments other structures can be coupled to and/or formed in the lower surface 211b of the base portion 210 of the housing 102. For example, the device 100 could include a rigid dome or other structure coupled to the lower surface 211b.

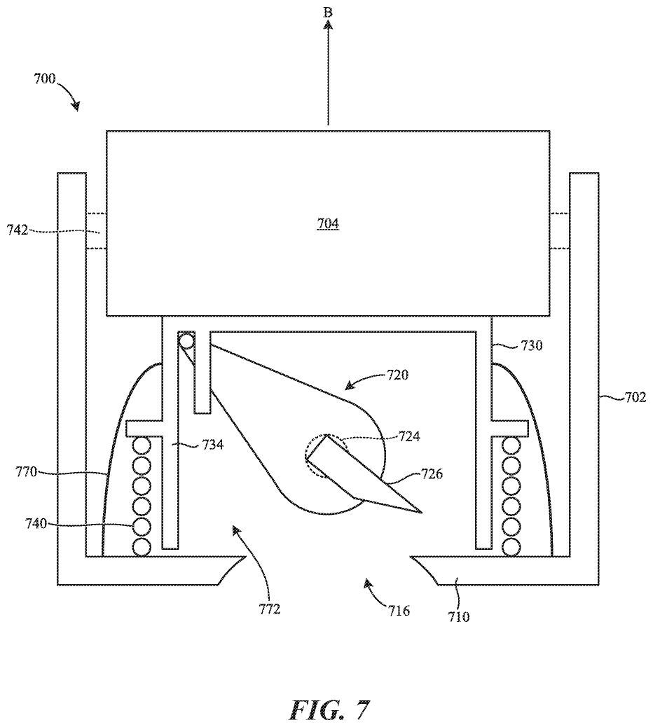

FIG. 7 is a partially schematic side cross-sectional view of a bodily fluid collection device 700 (“device 700”) configured in accordance with additional embodiments of the present technology. The device 700 can include features generally similar to and can operate generally similar to the device 100 described in detail with reference to FIGS. 1A-3D. For example, in the illustrated embodiment the device 700 includes a housing 702, an actuator 704, a skin-piercing assembly 720, and a plunger 730. The housing 702 includes a base portion 710 having an opening 716 configured to be positioned adjacent the skin of a patient. The actuator 704 is actuatable to unlock a locking mechanism 742 (shown schematically) and to release the plunger 730 which can be driven upward through the housing 702 by a first biasing member 740. Upward movement of the plunger 730 moves the plunger 730 out of engagement with the skin-piercing assembly 720. When the plunger 730 disengages the skin-piercing assembly 720, a second biasing member 724 can drive the skin-piercing assembly 720 to pivot such that a skin-piercing feature 726 sweeps at least partially through/along the opening 716 to pierce the skin of the patient.

In the illustrated embodiment, however, the device 700 includes a sealing member 770 (e.g., rather than the sealing member 244) positioned over the opening 716 and forming a lumen 772 within the device 700. In some embodiments, the sealing member 770 is coupled between the plunger 730 (e.g., a sidewall portion 734 of the plunger 730) and the housing 702 (e.g., the base portion 710 of the housing 702). The sealing member 770 can be a flexible membrane that can bend and/or is elastic. Accordingly, upward movement of the plunger 730 (e.g., in the direction of arrow B) can stretch the sealing member 770 and increase the volume of the lumen 772—thereby decreasing the pressure in the lumen 772 during use when the base portion 710 is sealed against the skin of the patient. As described in detail with reference to FIGS. 3A-3D and 6A-6C, this low pressure can act directly or indirectly against the skin of the patient to draw the skin toward/into the opening 716 to, for example, increase the draw volume of the device 700.

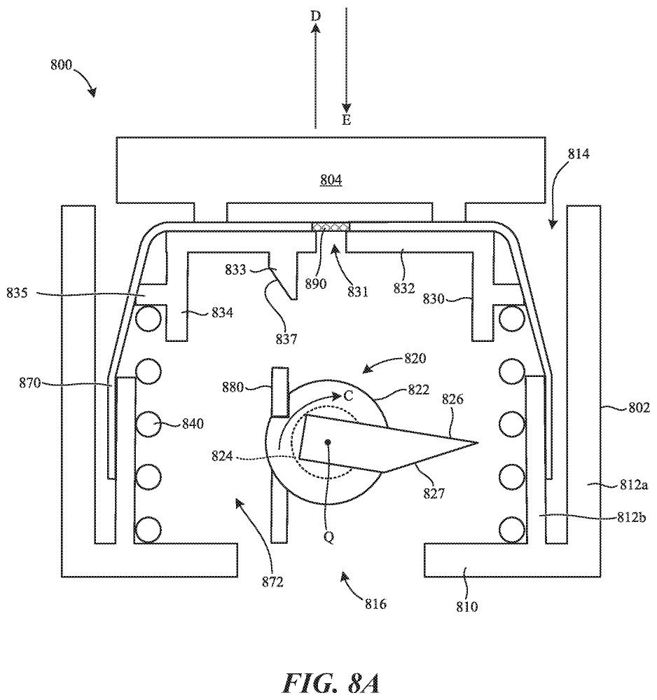

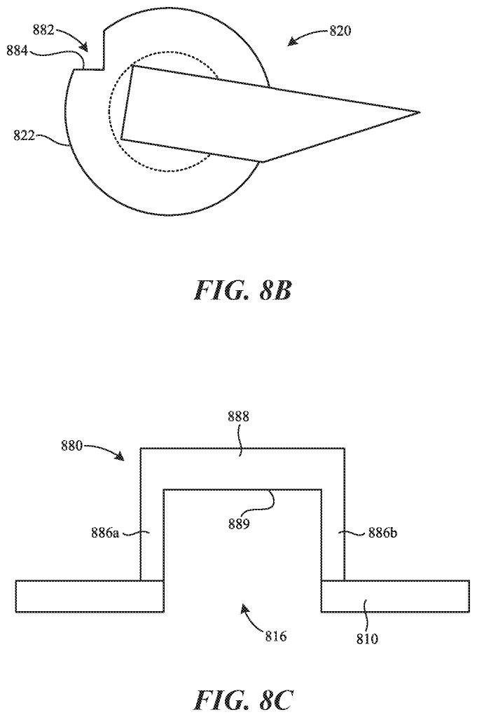

FIG. 8A is a side cross-sectional view of a bodily fluid collection device 800 (“device 800”) configured in accordance with additional embodiments of the present technology. The device 800 is in a pre-deployed configuration in FIG. 8A. The device 800 can include some features generally similar to and can operate generally similarly to the device 100 and/or the device 700 described in detail with reference to FIGS. 1A-7. For example, in the illustrated embodiment the device 800 includes a housing 802, an actuator 804, a skin-piercing assembly 820, and a plunger 830.

In the illustrated embodiment, the housing 802 includes a base portion 810, and a first sidewall portion 812a and a second sidewall portion 812b extending from the base portion 810 (e.g., extending generally perpendicular to and upward away from the base portion 210). The base portion 810 and the first sidewall portion 812a together define a lumen 814 in which the skin-piercing assembly 820 and the plunger 830 are at least partially positioned. During use of the device 800, a lower surface of the base portion 810 is configured to be positioned against and/or adjacent to the skin of the patient (e.g., the skin 101 shown in FIG. 1B). An opening 816 can extend through the base portion 810 such that the opening 816 abuts the skin 101 of the patient during use of the device 800. In some embodiments, as described in detail with reference to FIGS. 4 and 5, the opening 816 can be fluidly connected to one or more fluidic channels formed in, on, and/or through the base portion 810. During use of the device 800, the one or more fluidic channels can direct bodily fluid from the opening 816 to a reservoir and/or a detection site. In some embodiments, the opening 816 is configured (e.g., shaped and sized) to facilitate drawing of the skin 101 of the patient into and/or toward the lumen 814 during use of the device 800.

In the illustrated embodiment, the skin-piercing assembly 820 includes (i) a drive member 822 coupled to a first biasing member 824 (obscured in FIG. 8A and therefore shown in broken lines) and (ii) a skin-piercing feature 826 coupled to the drive member 822. In the illustrated embodiment, the skin-piercing feature 826 is a blade having a sharpened cutting edge 827. In other embodiments, the skin-piercing feature 826 is a needle, lancet, or other feature configured to pierce the skin 101 of the patient. In some embodiments, the first biasing member 824 couples the drive member 822 to the housing 802 such that the drive member 822 is rotatably/pivotably mounted within the lumen 814 of the housing 802 and configured to pivot about a pivot axis Q (e.g., in a direction indicated by arrow C). For example, the first biasing member 824 can be a torsion spring or other suitable biasing member that is connected between the drive member 822 and the second sidewall portion 812b of the housing 802 and/or another portion of the housing 802. In the illustrated embodiment, the device 800 further includes a retaining feature 880 configured to retain the skin-piercing assembly 820 in the pre-deployed configuration in which the first biasing member 824 is biased (e.g., wound).

More specifically FIG. 8B is a side view of the skin-piercing assembly 820 removed from the housing 804 and FIG. 8C is a rear view of the retaining feature 880 and the base portion 810 of the housing 802 shown in FIG. 8A in accordance with embodiments of the present technology. Referring to FIGS. 8A-8C together, the drive member 822 is generally circular and includes a notch 882 (e.g., a cut-out) including a notch surface 884. The retaining feature 880 can be generally U-shaped and include (i) a pair of legs 886 (identified individually as a first leg 886a and a second leg 886b) that extend from the base portion 810 of the housing 802 and (ii) a cross member 888 extending between the legs 886 and spanning across the opening 816 in the base portion 810. In the pre-deployed configuration illustrated in FIG. 8A, a lower surface 889 of the cross member 888 is configured to engage/contact the notch surface 884 of the drive member 822 to inhibit the drive member 822 (and the skin-piercing feature 826) from pivoting about the pivot axis Q in the direction indicated by the arrow C. In other embodiments, the skin-piercing assembly 820 and/or the retaining feature 880 can have different shapes, configurations, and the like such that the retaining feature 880 is configured to inhibit the skin-piercing assembly 820 from pivoting in the pre-deployed configuration.

Referring again to FIG. 8A, in the illustrated embodiment the plunger 830 is operably coupled to (i) the actuator 804 and (ii) a second biasing member 840 (e.g., a compression spring) configured to drive/bias the plunger 830 away from the base portion 810 of the housing 802 (e.g., in a direction indicated by arrow D). For example, the plunger 830 can include (i) a base portion 832 coupled to the actuator 804, (ii) a sidewall portion 834 extending from the base portion 832 (e.g., extending generally perpendicular to and downward away from the base portion 832), and (iii) a flange portion 835 projecting outwardly away from the sidewall portion 834. The second biasing member 840 can be coupled between the flange portion 835 and the base portion 810 of the housing 802.

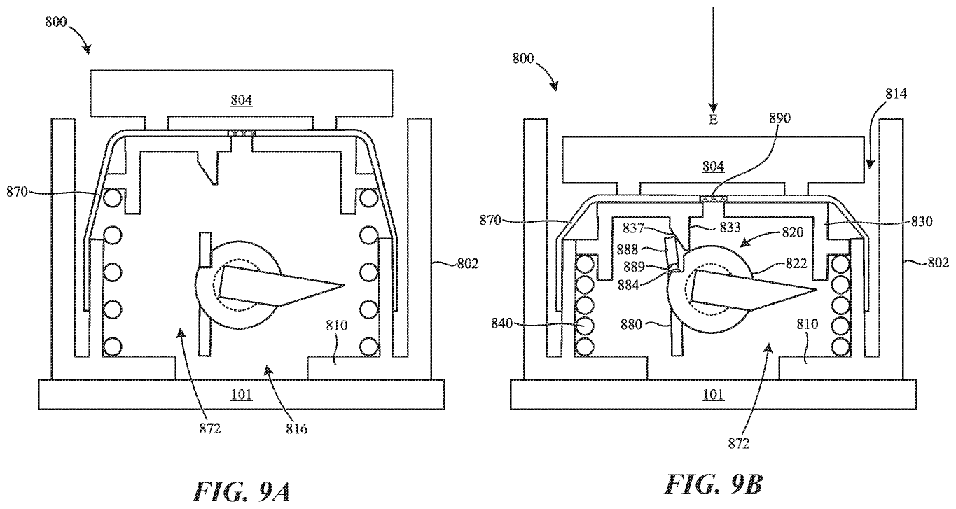

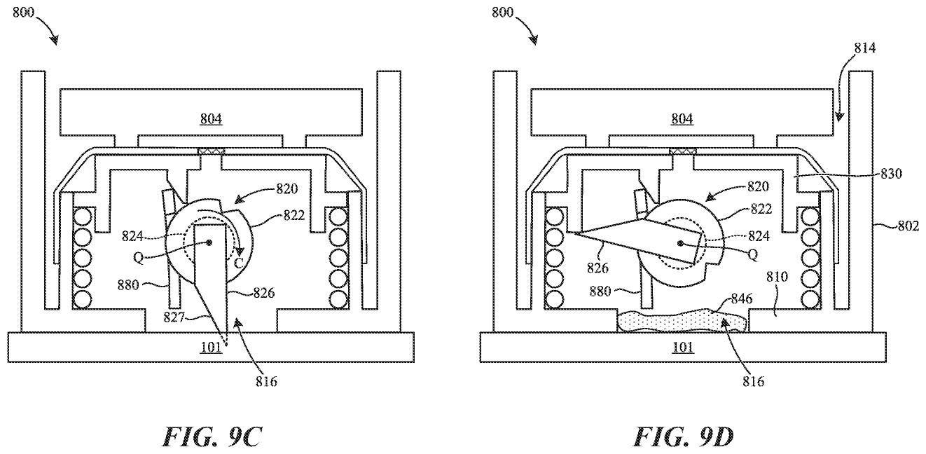

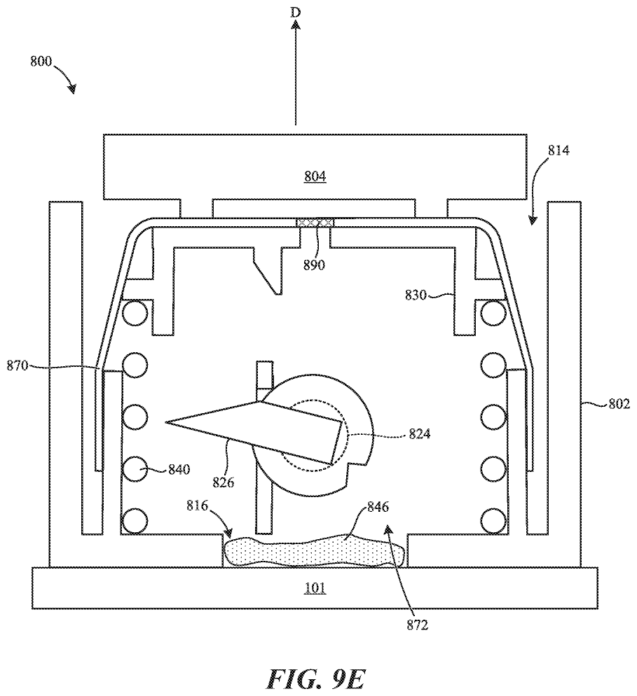

The plunger 830 can further include a projection 833 (e.g., an arm, release portion) extending downward away from the base portion 832 and having an angled release surface 837. As described in detail below with reference to FIGS. 9A-9E, during use of the device 800, the actuator 804 is configured to be pressed/depressed downward (e.g., in the direction indicated by the arrow E) to drive the plunger 830 through the lumen 814 of the housing 802 toward the base portion 810 of the housing 802 and against the biasing force of the second biasing member 840. As the actuator 804 is depressed, the release surface 837 of the projection 833 is configured to contact the retaining feature 880 (e.g., the cross member 888 shown in FIG. 8C) and to deflect the retaining feature 880 out of engagement with the drive member 822 of the skin-piercing assembly 820 so that the first biasing member 824 can drive the drive member 822 to pivot about the pivot axis Q. In some embodiments, the skin-piercing assembly 820 is translationally mounted within the lumen 814 of the housing 802 such that, when the actuator 804 is depressed, the skin-piercing assembly 820 engages and drives the skin-piercing assembly 820 toward the opening base portion 810 of the housing 802.

In the illustrated embodiment, the device 800 further includes a sealing member 870 coupled to the housing 802 (e.g., to the second sidewall portion 812b of the housing 802) and the plunger 830 and forming a lumen 872 within the device 800. The sealing member 870 can be a flexible membrane that can bend/stretch during movement of the plunger 830 to vary the volume of the lumen 872. In some embodiments, the device 800 can include a valve 890 coupled to the lumen 872 via, for example, an opening or hole 831 in the base portion 832 of the plunger 830. The valve 890 can be a one-way valve that permits air to escape from within the lumen 872 when the volume of the lumen 872 decreases (e.g., as the plunger 830 is moved in the direction of the arrow E), but inhibits air from entering the lumen 872 as the volume of the lumen 872 increases (e.g., as the second biasing member 840 drives the plunger 830 and the sealing member 870 away from the base portion 810 in the direction of the arrow D). During use of the device 800, the valve 890 can facilitate the creation a low-pressure region (e.g., a vacuum) within the lumen 872 that acts directly or indirectly against the skin 101 of the patient.

The various components of the device 800 can comprise metal, plastic, and/or other materials. For example, in some embodiments the housing 802, the actuator 804, the plunger 830, the drive member 822, and/or other components of the device 800 can be 3D-printed, molded (e.g., injection molded) or otherwise formed from a plastic material. In some embodiments, the device 800 can be manufactured to have the pre-deployed configuration shown in FIG. 8A.

FIGS. 9A-9E are side cross-sectional views of the device 800 illustrating various stages of a procedure to withdraw bodily fluid (e.g., blood) from a patient in accordance with additional embodiments of the present technology. Referring first to FIG. 9A, the device 800 is initially placed against the skin 101 of the patient in the pre-deployed configuration shown in FIG. 8A. More particularly, a lower surface of the base portion 810 of the housing 802 can be positioned against the skin 101 such that the opening 816 is adjacent to the skin 101. In some embodiments, the base portion 810 of the housing 802 can form a seal with the skin 101 of the patient such that the lumen 872 formed within the housing 802 by the sealing member 870 is sealed off from the environment around the device 800.

FIG. 9B shows the device 800 in a partially-deployed configuration after the actuator 804 has been depressed in the direction of the arrow E to drive the plunger 830 downward through the lumen 814 of the housing 802 toward the base portion 810. In some embodiments, the patient can press the actuator 804 downward using one or more of their fingers. In the illustrated embodiment, the projection 833 contacts the cross member 888 of the retaining feature 880 and deflects the retaining feature 880 out of engagement with the drive member 822 of the skin-piercing assembly 820. More particularly, the release surface 837 of the projection 833 can deflect the retaining feature 880 laterally such that the lower surface 889 of the cross member 888 no longer engages the notch surface 884 of the drive member 822. As further shown in FIG. 9B, depression of the actuator 804 causes the sealing member 870 to shrink/contract—reducing the volume in the sealed lumen 872. In some embodiments, air is driven out of the lumen 872 through the valve 890 as the actuator 804 is depressed. Moreover, depression of the actuator 804 compresses the second biasing member 840, which then exerts a biasing force against the plunger 830. In other embodiments, depression of the actuator 804 can also drive the skin-piercing assembly 820 partially through the lumen 814 toward the base portion 810 (e.g., by a predetermined distance).

When the skin-piercing assembly 820 is no longer restrained by the retaining feature 880, the first biasing member 824 drives the drive member 822 to pivot about the pivot axis Q (e.g., in the direction of the arrow C). FIG. 9C, for example, shows the device 800 in a deployed configuration in which the movement of the drive member 822 pivots the skin-piercing feature 826 downward toward/into the opening 816 and the skin 101. As shown, all or a portion of the cutting edge 827 can move through the opening 816 and can contact and incise the skin 101 positioned therein. The position of the skin-piercing assembly 820 relative to the opening 816 and the relative size and/or shape of the skin-piercing feature 826 can be varied to vary the size of the incision. In some aspects of the present technology, the drive member 822 is released from the retaining feature 880 instantaneously or nearly instantaneously, which can increase the velocity of the skin-piercing feature 826 to facilitate, for example, a cleaner incision, reduced patient pain, and/or a larger draw volume.

FIG. 9D shows the device 800 in a pre-retracted configuration after continued rotation of the skin-piercing assembly 820. In the illustrated embodiment, the first biasing member 824 has driven the drive member 822 around the pivot axis Q until (i) the skin-piercing feature 826 has swept past the opening 816 and no longer contacts the skin 101 of the patient and (ii) the skin-piercing feature 826 contacts the plunger 830. In other embodiments, the skin-piercing assembly 820 can be configured (e.g., shaped and sized) such that the skin-piercing feature 826 does not contact the plunger 830 in the pre-retracted configuration, contacts the retaining feature 880 in the pre-retracted configuration, and/or contacts another portion of the device 800 in the pre-retracted configuration. As shown in FIG. 9D, incising the skin 101 causes bodily fluid 946 (e.g., blood) to flow from the skin 101 into the device 800—for example, into the lumen 814 of the housing 102 and/or onto an upper surface of the base portion 810 of the housing 102. In some aspects of the present technology, the high rotational velocity of the skin-piercing feature 826 can increase the amount of the bodily fluid 946 withdrawn/collected while also making the procedure relatively painless for the patient.

FIG. 9E shows the device in a retracted configuration in which the second biasing member 840 has driven the plunger 830 upward in the direction of the arrow E through the lumen 814 of the housing 802. For example, the second biasing member 840 can drive the plunger 830 upward after the patient releases the actuator 804. As the plunger 830 moves upward, the volume within the sealed lumen 872 increases as the sealing member 870 expands/stretches. In some aspects of the present technology, increasing the volume within the lumen 872 generates reduced pressure (e.g., vacuum pressure) therein as the valve 890 does not let air into the lumen 872 during expansion of the lumen 872. In some embodiments, the reduced pressure can draw the skin 101 at least partially into the opening 816 (e.g., as shown in FIGS. 3B-3D). That is, the reduced pressure can deflect the skin 101 into the opening 816 and/or into the lumen 814 of the housing 802. Alternatively or additionally, the reduced pressure can help draw the bodily fluid 946 into the device 800. In some aspects of the present technology, the sealing member 870 and the valve 890 thereby increase the draw volume of the device 800. In some embodiments, the draw volume of the device 800 can be between 100-1000 μL (e.g., between 600-700 μL). In another aspect of the present technology, the device 800 is configured to draw the bodily fluid 946 in a relatively short period of time compared to conventional devices. For example, in some embodiments the device 800 can collect the draw volume in less than about 1 minute, less than about 45 seconds, less than about 30 seconds, or less than about 15 seconds.

In some embodiments, the device 800 is configured as a single-use device. For example, in the illustrated embodiment the device 800 is configured such that actuation of the actuator 804 when the device 800 is in the retracted configuration does not pivot the skin-piercing feature 826 into the opening 816. Specifically, the first biasing member 824 is no longer biased in the retracted configuration and therefore cannot drive the skin-piercing feature 826 toward the opening 816.

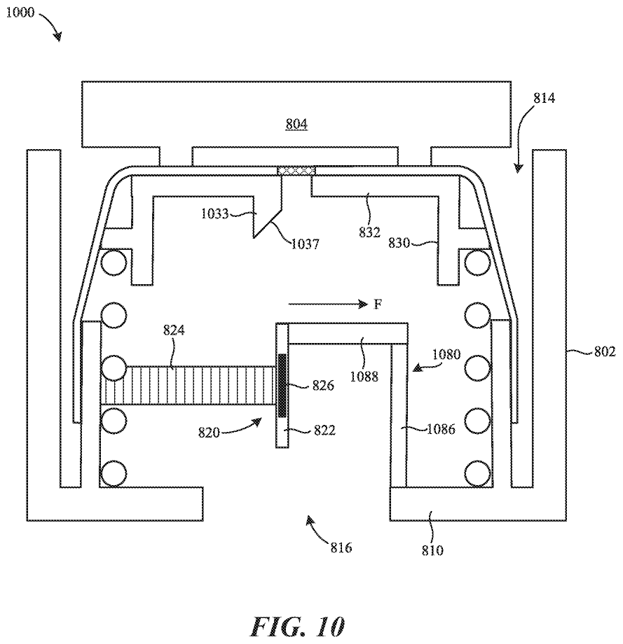

FIG. 10 is a side cross-sectional view of a bodily fluid collection device 1000 (“device 1000”) configured in accordance with additional embodiments of the present technology. The device 1000 is in a pre-deployed configuration in FIG. 10. The device 1000 can include some features generally similar to and can operate generally similarly to the device 800 described in detail with reference to FIGS. 8A-9E. For example, in the illustrated embodiment the device 1000 includes the housing 802, the actuator 804, the skin-piercing assembly 820, and the plunger 830.

In the illustrated embodiment, the device 1000 further includes a retaining feature 1080 (e.g., a shelf) configured to retain the skin-piercing assembly 820 in the pre-deployed configuration in which the first biasing member 824 is biased (e.g., wound). The retaining feature 1080 can include a vertical portion 1086 (e.g., a leg) that extends from the base portion 810 of the housing 802, and a horizontal portion 1088 (e.g., a cross member). In the pre-deployed configuration, the horizontal portion 1088 engages the notch 882 (FIGS. 8A and 8B) of the drive member 822 to inhibit the drive member 822 (and the skin-piercing feature 826) from pivoting into the opening 816.

In the illustrated embodiment, the plunger 830 includes a projection 1033 (e.g., an arm, release portion) extending downward away from the base portion 832 and having an angled release surface 1037. During use of the device 1000, the actuator 804 is configured to be pressed/depressed downward to drive the plunger 830 through the lumen 814 of the housing 802 toward the base portion 810 of the housing 802. As the actuator 804 is depressed, the release surface 1037 of the projection 1033 is configured to contact/engage the retaining feature 1080 (e.g., an edge portion of the horizontal portion 1088) and to deflect the retaining feature 1080 out of engagement with the drive member 822 of the skin-piercing assembly 820 so that the first biasing member 824 can drive the drive member 822 to pivot. More specifically, the projection 1033 can deflect the retaining feature 1080 in the direction indicated by arrow F out of the plane of rotation of the skin-piercing assembly 820. Thus, in comparison to the embodiment shown in FIGS. 8A-9E, the release surface 1037 is rotated (e.g., by about 90 degrees) relative to the release surface 837 of the projection 833. In some aspects of the present technology, this can help inhibit the retaining feature 1080 from interfering with the skin-piercing assembly 820 during rotation of the skin-piercing assembly 820. In other embodiments, the projection 1033 can be configured to contact and deflect the skin-piercing assembly 820 out of engagement with the (e.g., stationary) retaining feature 1080.

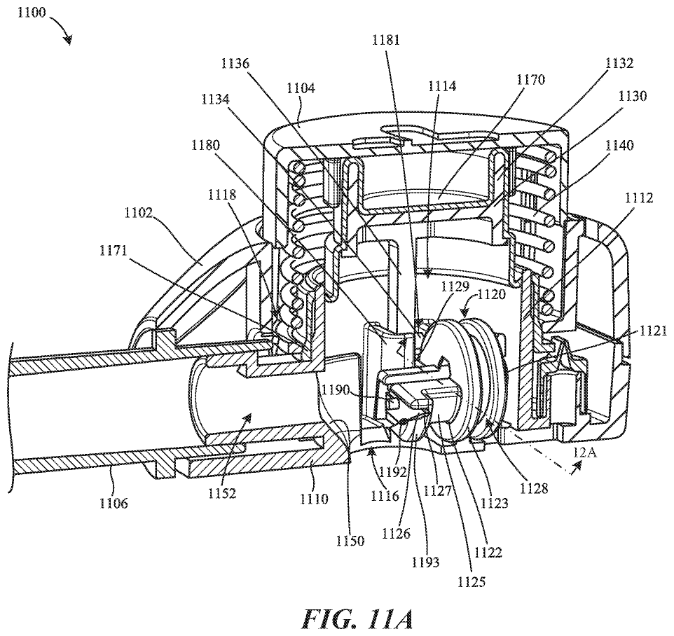

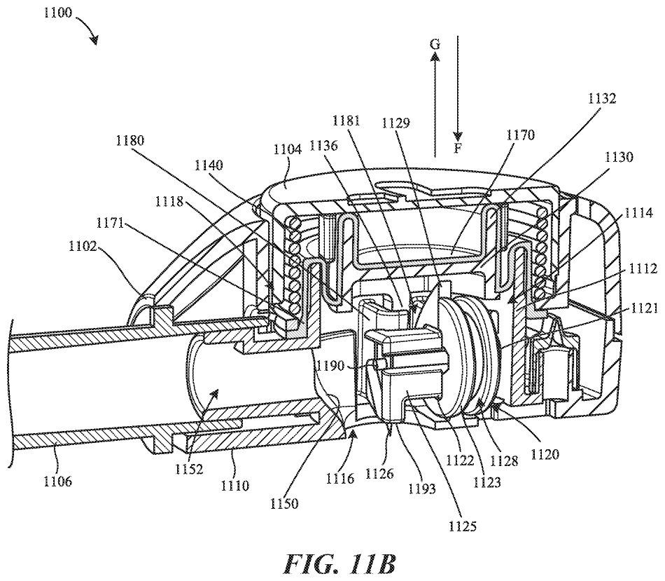

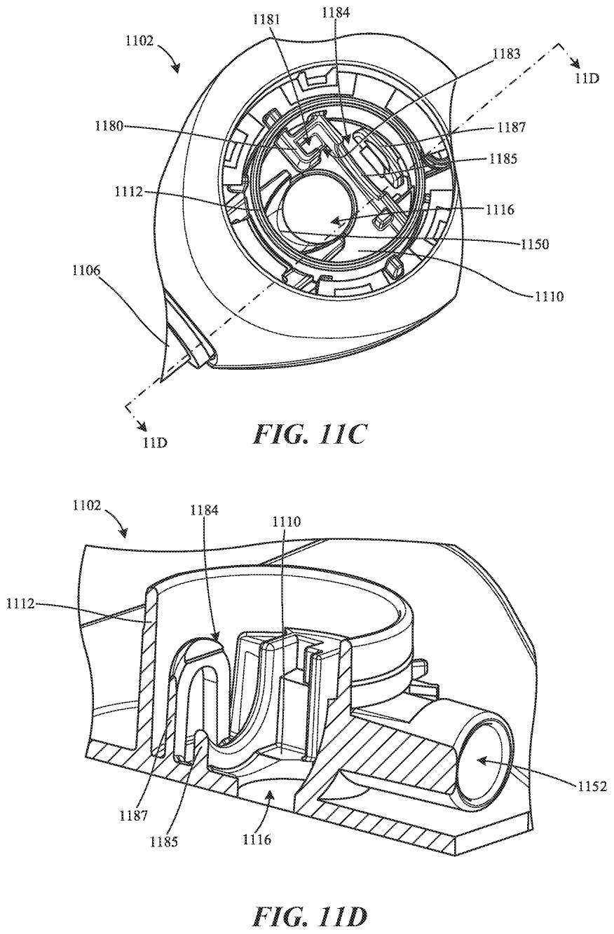

FIGS. 11A and 11B are partial cross-sectional side views of a bodily fluid collection device 1100 (“device 1100”) in a pre-deployment configuration (e.g., a pre-actuated position) and a deployed configuration (e.g., an actuated position), respectively, configured in accordance with additional embodiments of the present technology. The device 1100 can include some features generally similar to and/or can operate generally similarly to the devices 100, 700, 800, and/or 1000 described in detail with reference to FIGS. 1A-10. Referring to FIGS. 11A and 11B together, for example, the device 1100 includes a housing 1102, an actuator 1104, a skin-piercing assembly 1120 (not shown as cross-sectional in FIGS. 11A and 11B), a plunger 1130, and a sealing member 1170.

In the illustrated embodiment, the housing 1102 includes a base portion 1110 and a sidewall portion 1112 extending from the base portion 1110. The base portion 1110 and the sidewall portion 1112 together define a lumen 1114 in which the skin-piercing assembly 1120 and the plunger 1130 are at least partially positioned. During use of the device 1100, a lower surface of the base portion 1110 is configured to be positioned against and/or adjacent to the skin of the patient (e.g., the skin 101 shown in FIG. 1B). An opening 1116 can extend through the base portion 1110 such that the opening 1116 abuts the skin of the patient during use of the device 1100. The housing 1102 can further define a channel or groove 1118 extending about the sidewall portion 1112.

FIG. 11C is a top view of the housing 1102 in accordance with embodiments of the present technology, and FIG. 11D is a cross-sectional side view of the housing 1102 taken along the line 11D in FIG. 11C. Referring to FIGS. 11C and 11D together, the housing 1102 further includes a plunger guide 1180 and a skin-piercing assembly mount 1184 (e.g., a saddle) extending from and/or coupled to the base portion 1110. The plunger guide 1180 can include a wall or other structure that defines a recess 1181 and an opening 1183. The skin-piercing assembly mount 1184 can include a first portion 1185 and a second portion 1187 spaced apart from the first portion 1185. In the illustrated embodiment, the opening 1116 is fluidly connected to an outflow channel 1152 that extends through the sidewall via a fluidic channel 1150 formed in, on, and/or through the base portion 1110. A collection reservoir 1106 (omitted in FIG. 11D for clarity) can be releasably coupled to the outflow channel 1152 for receiving a flow of bodily fluid from the opening 1116 via the fluidic channel 1150.

Referring again to FIGS. 11A and 11B, the device 1100 can further include a first biasing member 1140 (e.g., a compression spring) positioned at least partially in the groove 1118 and operably coupled between the housing 1102 and the actuator 1104. In the pre-deployment position shown in FIG. 11A, the first biasing member 1140 is in a relaxed or unbiased state. To move the device 1100 to the deployed position shown in FIG. 11B, a user can push/depress the actuator 1104 downward toward the base portion 1110 against the biasing force of the first biasing member 1140. In some embodiments, an end portion 1171 of the sealing member 1170 can be secured to the housing 1102 in the groove 1118 such that the sealing member 1170 extends over and fluidly seals the lumen 1114.

In the illustrated embodiment, the plunger 1130 includes an upper portion 1132, a trigger portion 1134 (e.g., a lower portion; obscured in FIG. 11B), and an elongate middle portion 1136 extending between the upper portion 1132 and the trigger portion 1134. The trigger portion 1134 can be positioned at least partially within the recess 1181 of the plunger guide 1180. The upper portion 1132 can be operably coupled to the actuator 1104 and the sealing member 1170 such that downward movement (e.g., pressing) of the actuator 1104 drives the plunger 1130 toward the base portion 1110. More specifically, actuation of the actuator 1104 can drive the trigger portion 1134 of the plunger 1130 through the recess 1181. In other embodiments, the plunger 1130 can have other configurations (e.g., shapes, dimensions, couplings).

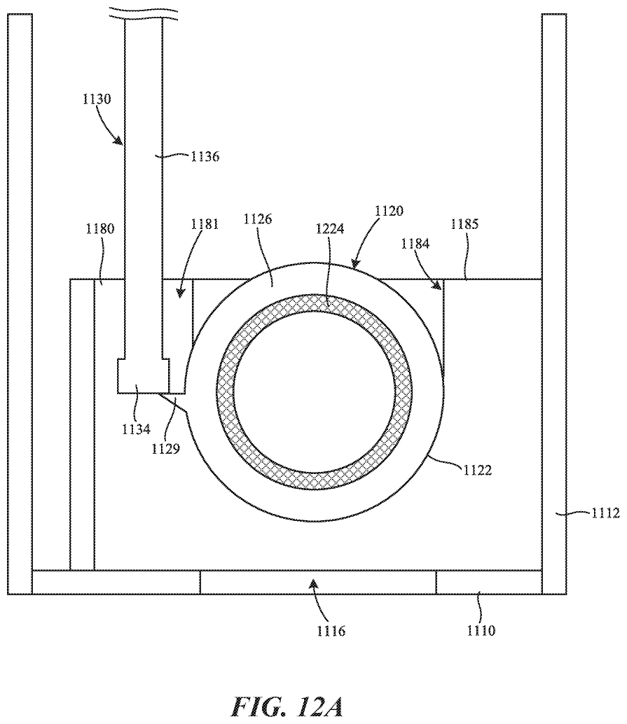

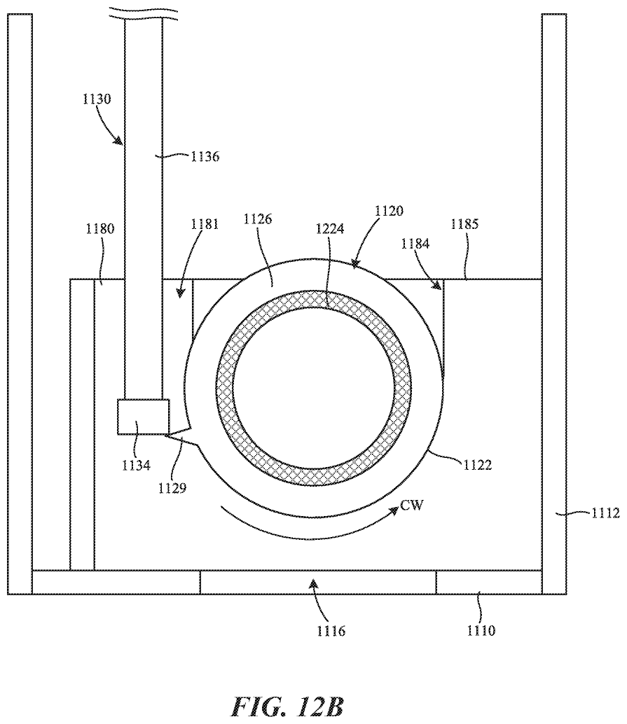

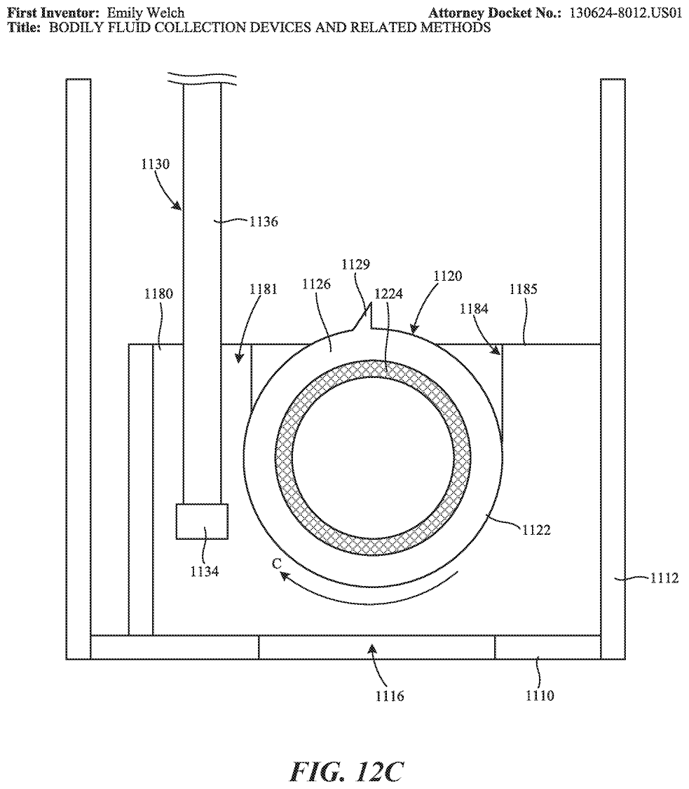

In the illustrated embodiment, the skin-piercing assembly 1120 includes a drive member 1122 (e.g., a drive body) having a retaining portion 1121 (partially obscured in FIGS. 11A and 11B), a mount portion 1123, and a hub portion 1125. The retaining portion 1121 and the mount portion 1123 can each have a circular cross-sectional shape, and the retaining portion 1121 can have a smaller diameter than the mount portion 1123. The mount portion 1123 can define an annular channel or groove 1128 extending at least partially thereabout (e.g., separated by a pair of adjacent ridges or flanges). With reference to FIGS. 11A-11D together, the drive member 1122 can be rotatably coupled to the skin-piercing assembly mount 1184. For example, the mount portion 1123 can be coupled to the first portion 1185 of the skin-piercing assembly mount 1184 by positioning (e.g., seating) the groove 1128 on/over the first portion 1185. In some embodiments the second portion 1187 of the skin-piercing assembly mount 1184 can engage the retaining portion 1121 to further secure the drive member 1122 to the housing 1102. In some embodiments, the drive member 1122 can be rotatably coupled to the housing 1102 via a second biasing member (obscured in FIGS. 11A and 11B but shown as second biasing member 1224 in FIGS. 12A-12C) such that the drive member 1122 is rotatably/pivotably mounted to the skin-piercing assembly mount 1184. For example, the second biasing member 1224 can be a torsion spring or other suitable biasing member that is connected between the drive member 1122 (e.g., the retaining portion 1121 and/or the mount portion 1123) and the sidewall portion 1112, the second portion 1187, and/or another portion of the housing 1102.

In the illustrated embodiment, the mount portion 1123 further includes a radially-projecting release member 1129 (e.g., a tab, bar, projection, fin). In the pre-deployment position shown in FIG. 11A, the release member 1129 is configured (e.g., shaped, sized, positioned) to extend through the opening 1183 in the plunger guide 1180 and into the recess 1181. As described in greater detail below with reference to FIGS. 12A-12C, the release member 1129 is configured to engage the trigger portion 1134 of the plunger 1130 in the pre-deployment position to inhibit rotation of the drive member 1122.

Referring again to FIGS. 11A and 11B, the skin-piercing assembly 1120 can further include a skin-piercing feature 1126 coupled to the hub portion 1125. In the illustrated embodiment, the skin-piercing feature 1126 is a blade having a sharpened cutting edge 1127 (obscured in FIG. 11B). In other embodiments, the skin-piercing feature 1126 can be a needle, lancet (e.g., cylindrical or other shaped lancet), or other feature configured to pierce the skin of a patient. In some embodiments, the skin-piercing assembly 1120 can include multiple skin-piercing features (e.g., multiple offset blades to produce a varied cutting pattern). In the illustrated embodiment, the skin-piercing feature 1126 is secured to the hub portion 1125 via a post 1190 and a bond 1192. The bond 1192 can comprise a weld, adhesive, press, and/or other bond and can be positioned at a portion of the cutting edge 1127. In some aspects of the present technology, positioning the bond 1192 at the cutting edge 1127 can help ensure that the skin-piercing feature 1126 does not move during operation—thereby ensuring a reliable cutting depth and length. In the illustrated embodiment, the hub portion 1125 further includes a cutting surface 1193, and the skin-piercing feature 1126 projects past the cutting surface 1193. During operation of the device 1100, the cutting surface 1193 can abut the skin of the patient. Accordingly, a distance that the skin-piercing feature 1126 projects past the cutting surface 1193 can define a maximum cutting depth of the skin-piercing assembly 1120 and can be adjusted/selected based on a desired cutting depth.

FIGS. 12A-12C are side cross-sectional views of the lumen 1114 of the device 1100 taken along the line 12A in FIG. 11A and illustrating the device 1100 in the pre-deployment position, a mid-deployment position, and the deployed position, respectively, in accordance with embodiments of the present technology. Referring first to FIGS. 11A and 12A together, in the pre-deployment position, the second biasing member 1224 is in a biased position (e.g., storing energy) and the release member 1129 of the mount portion 1123 engages the trigger portion 1134 of the plunger 1130 to inhibit rotation of the skin-piercing assembly 1120 by maintain the second biasing member 1224 in a biased position. To move the device 1100 to the deployed position to, for example, withdraw a bodily fluid from a patient, the base portion 1110 of the device 1100 can initially be placed against the skin of the patient with the device 1100 in the pre-deployment position. Then, the user can actuate (e.g., depress) the actuator 1104 in the direction of arrow F (FIG. 11B) against the biasing force of the first biasing member 1140.

Referring to FIGS. 11A, 11B, and 12B together, depressing the actuator 1104 drives the plunger 1130 and the sealing member 1170 downward through the lumen 1114 toward the base portion 1110. As the plunger 1130 moves downward, the trigger portion 1134 is driven through the recess 1181 of the plunger guide 1180. Initially, as shown in FIG. 12B, the trigger portion 1134 engages the release member 1129 and drives the drive member 1122 to rotate against the biasing force of the second biasing member 1224 in, for example, a counterclockwise direction indicated by the arrow CW. Accordingly, the downward movement of the plunger 1130 can increase the energy stored by the second biasing member 1224. Referring to FIGS. 11B and 12C together, upon continued depression of the plunger 1130, the release member 1129 disengages (e.g., slips by) the trigger portion 1134 allowing the second biasing member 1224 to drive the drive member 1122 to rotate in a clockwise direction indicated by the arrow C. As the drive member 1122 rotates, a portion of the cutting edge 1127 of the skin-piercing feature 1126 that extends past the cutting surface 1193 is driven through the opening 1116 and can contact and incise the skin positioned therein/therebelow.

Referring to FIGS. 11A and 11B and as noted above, the cutting surface 1193 can contact the skin and define a maximum size (e.g., depth, length) of the incision. In some embodiments, the configuration (e.g., a height or shape) of the skin-mount 11844 can be selected to position the skin-piercing assembly 1120 relative to the opening 1116 to further vary the size of the incision. In some embodiments, for example, the device 1100 can be configured to generate an incision that is between about 0.5-1.5 millimeters deep and about 3-7 millimeters long.

Similar to the embodiment described in detail above with reference to FIG. 3D, the skin-piercing assembly 1120 can continue to rotate to a retracted configuration in which the skin-piercing feature 1126 has been rotated past the opening 1116 and is secured within the lumen 1114. In the retracted configuration, the second biasing member 1224 has released at least a portion of its stored energy and the skin-piercing assembly 1120 no longer engages the plunger 1130 (e.g., via engagement of the trigger portion 1134 with the release member 1129). Accordingly, subsequent actuation of the actuator 1104 will not drive the skin-piercing feature 1126 out of the opening 1116.

In some embodiments, when the user releases the actuator 1104, the first biasing member 1140 can drive the plunger 1130, the sealing member 1170, and the actuator 1104 upward in the direction of arrow G (FIG. 11B). Moving the sealing member 1170 upward can increase the sealed volume in the lumen 1114 thereby generating vacuum pressure that can help draw the bodily fluid from the incision into the fluidic channel 1150.

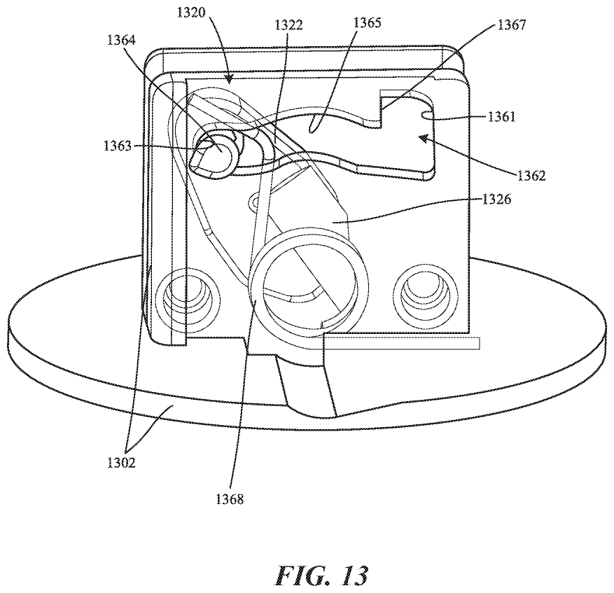

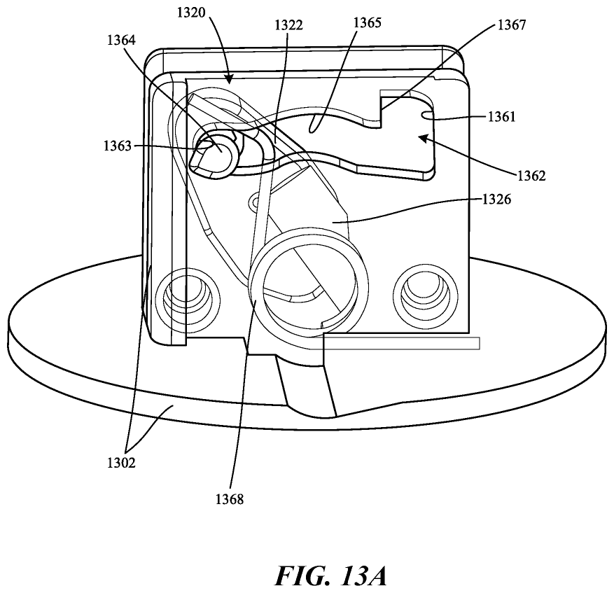

In other embodiments, bodily fluid collection devices configured in accordance with the present technology can have (i) skin-piercing assemblies that drive a blade or other skin-piercing feature through an opening in other manners and/or (ii) additional features for controlling the length and/or depth of an incision. For example, FIG. 13A is a partially transparent side view of a skin-piercing assembly 1320 coupled to a portion of a housing 1302 of a bodily fluid collection device in accordance with additional embodiments of the present technology. The housing 1302 is shown as partially transparent in FIG. 13A for clarity. In some embodiments, the skin-piercing assembly 1320 can (i) be incorporated into one or more of the devices 100, 700, 800, 1000, and/or 1100 described in detail with reference to FIGS. 1A-12C, and/or (ii) include features generally similar or identical to the skin-piercing assemblies 120, 720, 820, 1020, and/or 1120.

In the illustrated embodiment, the skin-piercing assembly 1320 includes a drive body 1322 having a projection 1364. A skin-piercing feature 1326 (e.g., a blade) can be coupled to the drive body 1322. The housing 1302 includes/defines a track 1362 (e.g., an opening, channel, elongate path), and the projection 1364 extends at last partially into the track 1362 such that the projection 1364 is confined to move along the track 1362. The track 1362 can extend between a first end portion 1361 and a second end portion 1363 and can have a length and shape (e.g., a varying elevation) selected to correspond to a desired path of the skin-piercing feature 1326 and a corresponding size (e.g., length, depth) of an incision created by the skin-piercing feature 1326. In the illustrated embodiment, for example, the track 1362 includes a raised middle portion 1365.

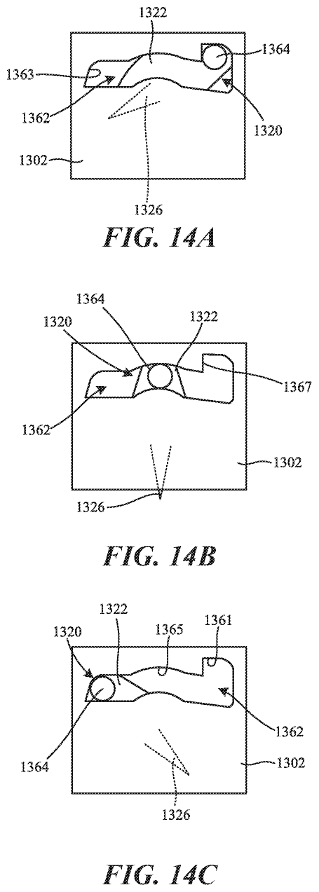

In some embodiments, the drive body 1322 is operably coupled to the housing 1302 via a biasing member 1368. In the illustrated embodiment, the biasing member 1368 is a torsion spring extending between the housing 1302 and the projection 1364, and the skin-piercing assembly 1320 is in a deployed position in which the torsion spring is in a relaxed state. FIGS. 14A-14C are side views of the housing 1302 and the skin-piercing assembly 1320 in a pre-deployment position, a mid-deployment position, and the deployed position, respectively, in accordance with embodiments of the present technology. Referring to FIGS. 13-14C together, the first end portion 1361 of the track 1362 can include a notch 1367 (e.g., a retaining surface, retaining feature) configured (e.g., shaped, sized) to engage the projection 1364 in the pre-deployment position such that the skin-piercing assembly 1320 is locked in the pre-deployment position with the biasing member 1368 in a biased state. The projection 1364 and/or another feature of the skin-piercing assembly 1320 can be operably coupled to an actuator or other feature of the bodily fluid collection device, and the actuator can be actuated to disengage the projection 1364 from the notch 1367. After actuation, the biasing member 1368 can drive the skin-piercing assembly 1320 along a path controlled/defined by the configuration (e.g., shape, size, length) of the track 1362 to deploy the skin-piercing feature 1326 through an opening in the housing (not shown) to, for example, incise the skin of a patient positioned therebelow.

In the illustrated embodiment, for example, the skin-piercing assembly 1320 moves laterally from the first end portion 1361 toward the second end portion 1363 while rotating to deploy the skin-piercing feature 1326 through the opening as the projection 1364 passes along the middle portion 1365. In some embodiments, the configuration of the middle portion 1365 can control a depth of an incision created by the skin-piercing feature 1326. For example, increasing the elevation of the middle portion 1365 can decrease the depth of the incision while, conversely, decreasing the elevation of the middle portion 1365 can increase the depth of the incision.

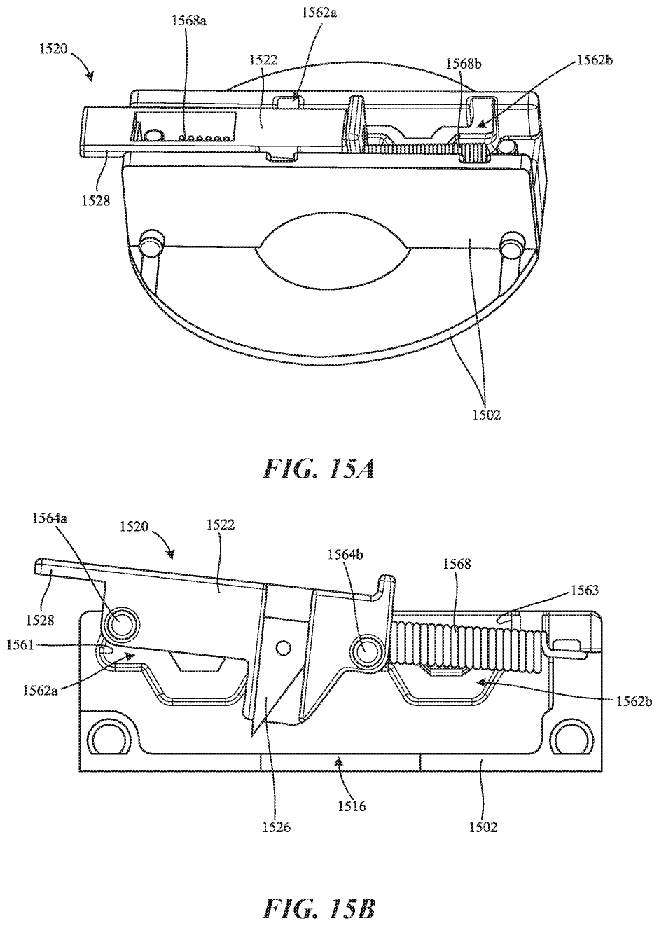

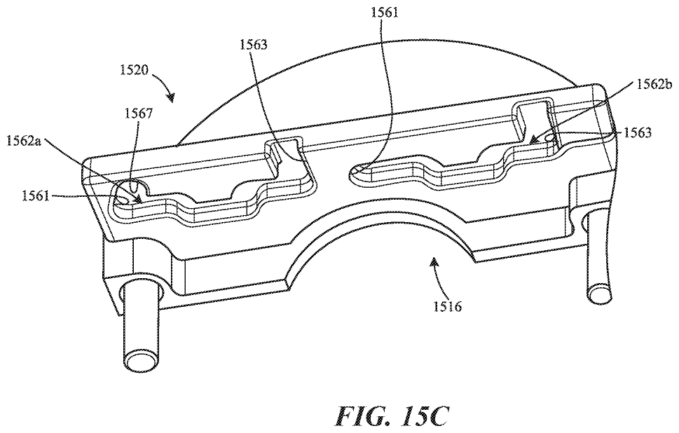

FIGS. 15A and 15B are a perspective side view and a cross-sectional side view, respectively, of a skin-piercing assembly 1520 coupled to a portion of a housing 1502 of a bodily fluid collection device configured in accordance with additional embodiments of the present technology. In some embodiments, the skin-piercing assembly 1520 can (i) be incorporated into one or more of the devices 100, 700, 800, 1000, and/or 1100 described in detail with reference to FIGS. 1A-12C, and/or (ii) include features generally similar or identical to the skin-piercing assemblies 120, 720, 820, 1020, 1120, and/or 1320.

In the illustrated embodiment, the skin-piercing assembly 1520 includes a drive body 1522 positioned at least partially over an opening 1516 in the housing 1502. A skin-piercing feature 1526 (e.g., a blade) can be coupled to the drive body 1522. FIG. 15C is a side cross-sectional view of the housing 1502 in accordance with embodiments of the present technology. Referring to FIGS. 15A-15C together, the housing 1502 can include/define a first track 1562a and a second track 1562b (e.g., openings, channels, elongate paths; collectively “tracks 1562”). The drive body 1522 of the skin-piercing assembly 1520 can include a first projection 1564a that extends at least partially into the first track 1562a and a second projection 1564b that extends at last partially into the second track 1562b (collectively “projections 1564”). The projections 1564 can be confined to move along the tracks 1562. The tracks 1562 can each have a configuration (e.g., length, shape) selected to correspond to a desired path of the skin-piercing feature 1526 and a corresponding size (e.g., length, depth) of an incision created by the skin-piercing feature 1526. For example, the tracks 1562 can each extend between and have a varying elevation between a first end portion 1561 and a second end portion 1563. The tracks 1562 can have the same or different configurations.

In some embodiments, the drive body 1522 includes a release member 1528 (e.g., a tab or projection) and is operably coupled to the housing 1502 via one or more biasing members 1568 (e.g., an individually identified first biasing member 1568a and a second biasing member 1568b). In the illustrated embodiment, the biasing members 1568 are extension springs, and the skin-piercing assembly 1520 is in a pre-deployment position in which the extension springs are in a biased state. In some embodiments, the first biasing member 1568a extends between the housing 1502 and a position of the drive body 1522 at or proximate the first projection 1564a, and the second biasing member 1568b extends between the housing 1502 and a position of the drive body 1522 at or proximate the second projection 1564b. In other embodiments, the drive body 1522 can be coupled to the housing via a single biasing member, or more than two biasing members.

In some embodiments, the first end portion 1561 of the first track 1562a can include a notch 1567 (e.g., a retaining surface, retaining feature) configured (e.g., shaped) to engage the first projection 1564a in the pre-deployment position such that the skin-piercing assembly 1520 is locked in the pre-deployment position with the biasing members 1568 in a biased state. The release member 1528 is configured to releasably engage a feature of a bodily fluid collection device—such as a restraint portion of a plunger (e.g., the restraint portion 238 shown in FIGS. 2-3D), a retaining feature (e.g., the retaining feature 880 shown in FIGS. 8A-9E and/or the retaining feature 1080 shown in FIG. 10), and/or the trigger portion of a plunger (e.g., the trigger portion 1136 shown in FIGS. 11A-12C). The bodily fluid collection device can be actuated to actuate (e.g., depress) the release member 1528 to unlock the projections 1564 from the tracks 1562. After the actuation, the biasing members 1568 can drive (e.g., pull) the skin-piercing assembly 1520 along the tracks 1562 from the first end portions 1561 toward the second end portions 1563 to deploy the skin-piercing feature 1526 through the opening 1516 to, for example, incise the skin of a patient positioned therebelow.

In other embodiments, the biasing members 1568 can be other types of biasing members configured to drive the skin-piercing assembly 1520. For example, the biasing members 1568 can be compression springs configured to push the skin-piercing assembly 1520 along the tracks 1562.

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A device for withdrawing bodily fluid from a patient, the device comprising:
a housing including a base having an opening extending therethrough;
a skin-piercing assembly positioned at least partially within the housing, wherein the skin-piercing assembly includes a skin-piercing feature and a biasing member; and
a plunger positioned at least partially within the housing and movable from a first position to a second position, wherein—
in the first position, the plunger is configured to engage the skin-piercing assembly to maintain the biasing member in a biased configuration, and
movement of the plunger from the first position to the second position disengages the plunger from the skin-piercing assembly to permit the biasing member to drive the skin-piercing feature at least partially through the opening in the base.

2. The device of claim 1, further comprising an actuator operably coupled to the plunger, wherein movement of the actuator in a direction toward the base drives the plunger from the first position to the second position.

3. The device of claim 2 wherein the biasing member is a first biasing member, and further comprising a second biasing member operably coupled between the actuator and the housing, wherein the second biasing member is configured to bias the plunger to the first position.

4. The device of claim 3 wherein the first biasing member is a torsion spring, and wherein the second biasing member is a compression spring.

5. The device of claim 1 wherein the plunger includes a trigger portion, wherein the skin-piercing assembly includes a drive member having a projection, and wherein the trigger portion engages the projection in the first position to maintain the biasing member in the biased configuration.

6. The device of claim 5 wherein movement of the plunger from the first position to the second position (a) drives the trigger portion against the projection against the biasing force of the biasing member and then (b) drives the trigger portion past the projection to permit the biasing member to drive the skin-piercing feature at least partially through the opening in the base.

7. The device of claim 1 wherein the biasing member is a first biasing member, and further comprising a second biasing member operably coupled between the plunger and the housing, wherein the second biasing member is configured to drive the plunger from the first position to the second position.

8. The device of claim 7 wherein the second position is farther away from the base than the first position.

9. The device of claim 7, further comprising a locking mechanism operably coupled to the plunger and configured to selectively maintain the second biasing member in a biased configuration.

10. The device of example 9, further comprising a release actuator operably coupled to the locking mechanism, wherein actuation of the release actuator unlocks the locking mechanism to permit the second biasing member to drive the plunger from the first position to the second position.

11. The device of claim 1, further comprising a flexible sealing member coupled to the housing and positioned to define a sealed volume within the housing.

12. The device of claim 1 wherein the biasing member is a torsion spring.

13. The device of claim 1 wherein the skin-piercing feature is a blade.

14. The device of claim 1 wherein the biasing member is configured to rotate the skin-piercing feature relative to the housing.

15. The device of claim 1 wherein the skin-piercing assembly further includes a drive body, wherein the biasing member is operably coupled between the drive body and the housing, and wherein the skin-piercing feature is coupled to the drive body.

16. The device of claim 15 wherein the skin-piercing feature is a blade having a cutting edge, wherein a portion of the cutting edge is directly bonded to the drive body.

17. The device of claim 1, further comprising a flexible membrane coupled to the base of the housing across the opening, wherein, in the second position, the plunger is configured to disengage the skin-piercing assembly to permit the biasing member to drive the skin-piercing feature at least partially through the opening in the base to puncture the flexible membrane.

18. The device of claim 1, further comprising:
a reservoir releasably coupled to the housing; and
a fluidic channel configured to direct bodily fluid from the opening to the reservoir.

19. A device for withdrawing bodily fluid from a patient, the device comprising:
a housing including a base having an opening extending therethrough;
a skin-piercing assembly positioned at least partially within the housing, wherein the skin-piercing assembly includes a drive member, a blade coupled to the drive member, and a biasing member;
an actuator movable relative to the housing;
a plunger rotatably coupled to the housing and having a pre-deployment position, wherein—
in the pre-deployment position, the plunger engages the drive member to inhibit rotation of the blade, and
movement of the actuator in a direction toward the base disengages the plunger from the drive member to permit the first biasing member to rotate the blade at least partially through the opening in the base.

20. A device for withdrawing bodily fluid from a patient, the device comprising:
a housing including a base having an opening extending therethrough;
a skin-piercing assembly positioned at least partially within the housing, wherein the skin-piercing assembly includes a skin-piercing feature and a biasing member;
a retaining feature configured to engage the skin-piercing assembly to maintain the biasing member in a biased configuration; and
a plunger positioned at least partially within the housing, wherein the plunger is movable through the housing to engage the retaining feature to deflect the retaining feature out of engagement with the skin-piercing assembly to permit the biasing member to drive the skin-piercing feature at least partially through the opening in the base.
